(19) United States
(12) Patent Application Publication
(10) Pub. No.: US 2025/0320291 A1
(43) Pub. Date: Oct. 16, 2025

(54) COMPOSITIONS AND USES OF CD19 TARGETED CHIMERIC ANTIGEN RECEPTOR MODIFIED IMMUNE CELLS

(71) Applicant: City of Hope, Duarte, CA (US)

(72) Inventors: Christine E. Brown, Duarte, CA (US); Lawrence Stern, Duarte, CA (US); Stephen J. Forman, Duarte, CA (US); Xiuli Wang, Temple City, CA (US); John Williams, Duarte, CA (US)

(21) Appl. No.: 18/266,443

(22) PCT Filed: Dec. 9, 2021
(86) PCT No.: PCT/US2021/062708

§ 371 Date: Jun. 9, 2023

Related U.S. Application Data
(60) Provisional application No. 63/123,433, filed on Dec. 9, 2020.

(51) Int. Cl. C07K 16/28; C07K 14/725

(52) U.S. Cl. CPC C07K 16/2803; C07K 14/7051; C07K 2317/622; C07K 2319/03

(57) ABSTRACT

CD19 scFv, methods for using such scFv, CAR that include such scFv, methods for making and using such CD19 targeted CAR T cells and CD19 targeted CAR natural killer (NK) cells are described as a methods to treat a variety of cancers.

### CLAIM OF PRIORITY

This application is a National Stage application under 35 U.S.C. § 371 of International Application No. PCT/US2021/062708, filed on Dec. 9, 2021, which claims the benefit of U.S. Provisional Application Ser. No. 63/123,433, filed on Dec. 9, 2020. The entire contents of the foregoing are incorporated herein by reference.

### STATEMENT OF FEDERALLY SPONSORED RESEARCH

This invention was made with government support under P50 CA107399 awarded by the National Institutes of Health. The government has certain rights in the invention.

### SEQUENCE LISTING

This application contains a Sequence Listing that has been submitted electronically as an ASCII text file named 40056-0060US1_ST25.txt. The ASCII text file, created on May 8, 2024, is 109,212 bytes in size. The material in the ASCII text file is hereby incorporated by reference in its entirety.

### TECHNICAL FIELD

This disclosure concerns CD19-specific chimeric antigen receptor (CAR)-engineered immune cells, methods of formulating, and methods of use.

### BACKGROUND

Chimeric antigen receptor (CAR) engineered T cells have energized the field of cancer immunotherapy with their proven ability to treat CD19+ malignancies in the clinic and emerging efficacy in treating other diseases. The CAR antigen recognition domain often requires considerable engineering to generate binding interactions resulting in target-specific activation and there is a need for additional highly functional antigen recognition domains.

### SUMMARY

Described herein are two CD19-targeted scFv (“CD19 scFv”) developed by an approach that included protein engineering and combinatorial library screening to facilitate scFv humanization and affinity modulation. Development of the scFv employed yeast surface display, a genotype-phenotype linkage strategy for functional screening of proteins of interest through protein expression and tethering to the yeast cell wall by covalent linkage. This linkage allows for facile screening of large combinatorial libraries. Importantly, the screening employed to develop the CD19 scFv described herein used linkers similar to those used in CAR constructs. The use of CAR-like linkers in scFv screening increases the likelihood that the isolated scFvs will be functional in the desired molecular context, e.g., N- or C-terminal linkage of the scFv and flexibility of the spacer region.

Two computational humanization methods were applied to the antigen recognition domain of a previously developed CD19-targeted CAR and yeast surface display techniques were applied to screen expression, foldedness, and affinity of the humanized variants. The resulting CARs showed comparable activity to the equivalent murine versions in degranulation and internal cytokine staining assays.

Described herein are CD19 scFv, methods for using such scFv, CAR that include such scFv, methods for making and using such CD19 targeted CAR T cells (also herein called CD19 CAR T cells) and CD19 targeted CAR natural killer (NK) cells (also herein called CD19 CAR NK cells) to treat a variety of cancers (collectively CD19 CAR cells).

Described herein is a method of treating a proliferative disease (e.g., a cancer or malignancy or a precancerous condition) associated with the unwanted expression of CD19 on cells. Thus, the methods include treating: a myelodysplasia, a chronic or acute leukemia or lymphoma, e.g., a relapsed and/or refractory lymphoma, a relapsed and/or refractory leukemia. In some cases, the patient is suffering from: B-cell acute lymphoid leukemia (BALL), T-cell acute lymphoid leukemia (TALL), small lymphocytic leukemia (SLL), acute lymphoid leukemia (ALL), relapsing and refractory ALL, B cell prolymphocytic leukemia, chronic myelogenous leukemia (CML), and chronic lymphocytic leukemia (CLL), mantle cell lymphoma (MCL), blastic plasmacytoid dendritic cell neoplasm, Burkitt's lymphoma, follicular lymphoma, hairy cell leukemia, small cell- or a large cell-follicular lymphoma, diffuse large B cell lymphoma (DLBCL), MALT lymphoma, marginal zone lymphoma, multiple myeloma, myelodysplasia and myelodysplastic syndrome, non-Hodgkin lymphoma, Hodgkin lymphoma, plasmablastic lymphoma, and Hodgkin lymphoma. The methods entail administering to a patient in need thereof a population of autologous or allogeneic human immune cells (e.g., T cell or NK cells) comprising a nucleic acid molecule encoding a polypeptide or CAR described herein (e.g., a vector or mRNA comprising such a nucleic acid molecule.

Also described herein are methods for using CD19 CAR T cells or CD19 CAR NK cells as anti-cancer agents selective against CD19-positive cells.

Described herein is a nucleic acid molecule comprising a nucleotide sequence encoding a chimeric antigen receptor (CAR) or polypeptide, wherein the chimeric antigen receptor or polypeptide comprises: an scFv targeting CD19, a spacer, a transmembrane domain, a co-stimulatory domain, and a CD3ζ signaling domain.

In various embodiments: the transmembrane domain is selected from: a CD4 transmembrane domain or variant thereof having 1-5 amino acid modifications, a CD8 transmembrane domain or variant thereof having 1-5 amino acid modifications, a CD28 transmembrane domain or a variant thereof having 1-5 amino acid modifications; the spacer comprises 20-150 amino acids and is located between the scFv and the transmembrane domain; the transmembrane domain is a CD4 transmembrane domain or variant thereof having 1-5 amino acid modifications; the transmembrane domain is a CD4 transmembrane domain; the chimeric antigen receptor comprises a transmembrane domain selected from: a CD4 transmembrane domain or variant thereof having 1-2 amino acid modifications, a CD8 transmembrane domain or variant thereof having 1-2 amino acid modifications, a CD28 transmembrane domain or a variant thereof having 1-2 amino acid modifications; the spacer region comprises an amino acid sequence selected from the group consisting of SEQ ID NOs: 2-12 or a variant thereof having 1-5 amino acid modifications; the spacer comprises an IgG hinge region; the spacer comprises 10-50 amino acids; the costimulatory domain comprises the amino acid sequence of SEQ ID NO: 22, 23, or 24 or a variant thereof having 1-5 amino acid modifications; the CD3ζ signaling domain comprises the amino acid sequence of SEQ ID NO:21; a linker of 3 to 15 amino acids is located between the costimulatory domain and the CD3ζ signaling domain or variant thereof; the CAR or polypeptide comprises the amino acid sequence of SEQ ID NO: 1 or a variant thereof having 1-5 amino acid modifications; the scFv comprises the amino acid sequence of SEQ ID NO:1.

Also disclosed herein is: a viral vector comprising a nucleic acid molecule described herein; a population of human T cells (e.g., a population comprising central memory T cells) or of human NK cells transduced by a vector comprising a nucleic acid molecule described herein. In some embodiments, the T cells comprise PBMC, dPBMC (PBMC with depletion of CD14+ and CD25+ cells), Tn/mem (naïve and memory T cells, CD62L+ enriched from dPBMC), or Tcm (central memory T cells).

In one embodiment: the chimeric antigen receptor or the polypeptide comprises: a CD19 scFv, e.g., an scFv comprising the amino acid sequence: DIQMTQSPSSLSASVGDRVTITCRASQDISKYLNWYQQKPGGAPKLLIYHTSRLHSGV PSRFSGSGSGTDFTFTISSLQQEDIATYYCQQGNTLPYTFGQGTKLEIKGSTSGSGKPG SGEGSTKGQVQLQESGPGLVAPSQTLSLTCTVSGVSLPDYGVSWIRQPPRKGLEWIGV IWGSETTYYNSALKSRVTISVDNSKNQFSLKLSSVTAADTAVYYCAKHYYYGGSYA MDYWGQGTLVTVSS (SEQ ID NO: 1) with up to 5 or up to 10 single amino acid substitutions). This scFv is referred to as VH4Vκ1.

In certain embodiments, the CD19 scFv comprises a light chain variable region that is at least 95% identical to or includes up to 5 single amino acid substitutions compared to: DIQMTQSPSSLSASVGDRVTITCRASQDISKYLNWYQQKPGGAPKLLIYHTSRLHSGV PSRFSGSGSGTDFTFTISSLQQEDIATYYCQQGNTLPYTFGQGTKLEIKGST (SEQ ID NO: 50). In certain embodiments, the CD19 scFv comprises a light chain variable region that comprises a CDR1 comprising: RASQDISKYLN (SEQ ID NO: 51), a CDR2 comprising HTSRLHS (SEQ ID NO: 52); and a CDR3 comprising QQGNTLPYT (SEQ ID NO: 53) and is overall at least 97, 98 or 99% identical to SEQ ID NO: 50).

In certain embodiments, the CD19 scFv comprises a heavy chain variable region that is at least 95% identical to or includes up to 5 single amino acid substitutions compared to: QVQLQESGPGLVAPSQTLSLTCTVSGVSLPDYGVSWIRQPPRKGLEWIGVIWGSETT YYNSALKSRVTISVDNSKNQFSLKLSSVTAADTAVYYCAKHYYYGGSYAMDYWGQ GTLVTVSS (SEQ ID NO: 54). In certain embodiments, the CD19 scFv comprises a heavy chain variable region that comprises a CDR1 comprising: DYGVS (SEQ ID NO: 57), a CDR2 comprising VIWGSETTYYNSALKS (SEQ ID NO: 60); and a CDR3 comprising HYYYGGSYAMDY (SEQ ID NO: 59) and is overall at least 97, 98 or 99% identical to SEQ ID NO: 54).

In certain embodiments, the CD19 scFv comprises a light chain variable region comprising DIQMTQSPSSLSASVGDRVTITCRASQDISKYLNWYQQKPGGAPKLLIYHTSRLHSGV PSRFSGSGSGTDFTFTISSLQQEDIATYYCQQGNTLPYTFGQGTKLEIKGST (SEQ ID NO: 50) and a heavy chain variable region comprising QVQLQESGPGLVAPSQTLSLTCTVSGVSLPDYGVSWIRQPPRKGLEWIGVIWGSETT YYNSALKSRVTISVDNSKNQFSLKLSSVTAADTAVYYCAKHYYYGGSYAMDYWGQ GTLVTVSS (SEQ ID NO: 54) joined by a linker of 5-20 amino acids. In some embodiments, a useful flexible linker is 1, 2, 3, 4, 5, 6, 7, 8, 9, or 10 repeats of the sequence GGGS (SEQ ID NO:34). In some embodiments, a useful flexible linker is 1, 2, 3, 4, 5, 6, 7, 8, 9, or 10 repeats of the sequence GGGGS (SEQ ID NO:35). Preferably the light chain variable region is amino terminal to the heavy chain variable region.

In another embodiment: the chimeric antigen receptor or the polypeptide comprises: a CD19 scFv, e.g., an scFv comprising the amino acid sequence: DIQMTQSPSSLSASVGDRVTITCRASQDISKYLNWYQQKPGKAPKLLIYHTSRLHSGV PSRFSGSRSGTDFTLTISSLQPEDFATYYCQQGNTLPYTFGQGTKVEIKGSTSGSGKPG SGEGSTKGEVQLVESGGGLVQPGGSLRLSCAASGVSLPDYGVSWVRQAPGKGLEW VAVIWGSETTYYADSVKGRFTISADTSKNTAYLQMNSLRAEDTAVYYCSRHYYYGG SYAMDYWGQGTLVTVSS (SEQ ID NO:32) with up to 5 or up to 10 single amino acid substitutions). This scFv is referred to as 4D5.

In certain embodiments, the CD19 scFv comprises a light chain variable region that is at least 95% identical to or includes up to 5 single amino acid substitutions compared to: DIQMTQSPSSLSASVGDRVTITCRASQDISKYLNWYQQKPGKAPKLLIYHTSRLHSGV PSRFSGSRSGTDFTLTISSLQPEDFATYYCQQGNTLPYTFGQGTKVEIKGST (SEQ ID NO: 55) or (SEQ ID NO: 50). In certain embodiments, the CD19 scFv comprises a light chain variable region that comprises a CDR1 comprising: RASQDISKYLN (SEQ ID NO: 51), a CDR2 comprising HTSRLHS (SEQ ID NO: 52); and a CDR3 comprising QQGNTLPYT (SEQ ID NO: 53) and is overall at least 97, 98 or 99% identical to SEQ ID NO: 55).

In certain embodiments, the CD19 scFv comprises a heavy chain variable region that is at least 95% identical to or includes up to 5 single amino acid substitutions compared to: EVQLVESGGGLVQPGGSLRLSCAASGVSLPDYGVSWVRQAPGKGLEWVAVIWGSE TTYYADSVKGRFTISADTSKNTAYLQMNSLRAEDTAVYYCSRHYYYGGSYAMDYW GQGTLVTVSS (SEQ ID NO: 56). In certain embodiments, the CD19 scFv comprises a heavy chain variable region that comprises a CDR1 comprising: DYGVS (SEQ ID NO: 57), a CDR2 comprising VIWGSETTYYADSVKG (SEQ ID NO: 58); and a CDR3 comprising HYYYGGSYAMDY (SEQ ID NO: 59) and is overall at least 97, 98 or 99% identical to SEQ ID NO: 56).

In certain embodiments, the CD19 scFv comprises a light chain variable region comprising DIQMTQSPSSLSASVGDRVTITCRASQDISKYLNWYQQKPGKAPKLLIYHTSRLHSGV PSRFSGSRSGTDFTLTISSLQPEDFATYYCQQGNTLPYTFGQGTKVEIKGST (SEQ ID NO: 55) and a heavy chain variable region comprising EVQLVESGGGLVQPGGSLRLSCAASGVSLPDYGVSWVRQAPGKGLEWVAVIWGSE TTYYADSVKGRFTISADTSKNTAYLQMNSLRAEDTAVYYCSRHYYYGGSYAMDYW GQGTLVTVSS (SEQ ID NO: 56) joined by a linker of 5-20 amino acids. In some embodiments, a useful flexible linker is 1, 2, 3, 4, 5, 6, 7, 8, 9, or 10 repeats of the sequence GGGS (SEQ ID NO:34). In some embodiments, a useful flexible linker is 1, 2, 3, 4, 5, 6, 7, 8, 9, or 10 repeats of the sequence GGGGS (SEQ ID NO:35). Preferably the light chain variable region is amino terminal to the heavy chain variable region.

Also described are T cells or NK cells harboring a vector expressing the CAR or the polypeptide. In various embodiments: at least 20%, 30%, or 40% of the transduced human T cells are central memory T cells; at least 30% of the transduced human T cells are CD4+ and CD62L+ or CD8+ and CD62L+; the population of human T cells are autologous to the patient; and the population of human T cells are allogenic to the patient.

### CD19 Targeted CAR

The CD19 targeted CAR (also called “CD19 CAR”) or CD19 targeted polypeptide (also called “CD19 polypeptide”) described herein include a CD19 targeting scFv. In some embodiments, an scFv comprising the amino acid sequence: DIQMTQSPSSLSASVGDRVTITCRASQDISKYLNWYQQKPGGAPKLLIYHTSRLHSGV PSRFSGSGSGTDFTFTISSLQQEDIATYYCQQGNTLPYTFGQGTKLEIKGSTSGSGKPG SGEGSTKGQVQLQESGPGLVAPSQTLSLTCTVSGVSLPDYGVSWIRQPPRKGLEWIGV IWGSETTYYNSALKSRVTISVDNSKNQFSLKLSSVTAADTAVYYCAKHYYYGGSYA MDYWGQGTLVTVSS (SEQ ID NO:1) or comprising the sequence

(SEQ ID NO: 32)
DIQMTQSPSSLSASVGDRVTITCRASQDISKYLNWYQQKPGKAPKLLIYH
TSRLHSGVPSRFSGSRSGTDFTLTISSLQPEDFATYYCQQGNTLPYTFGQ
GTKVEIKGSTSGSGKPGSGEGSTKGEVQLVESGGGLVQPGGSLRLSCAAS
GVSLPDYGVSWVRQAPGKGLEWVAVIWGSETTYYADSVKGRFTISADTSK
NTAYLQMNSLRAEDTAVYYCSRHYYYGGSYAMDYWGQGTLVTVSS.

A useful CD19 CAR or CD19 polypeptide can consist of or comprises the amino acid sequence of SEQ ID NO:30 or 33 (mature CAR lacking a signal sequence); a useful CD19 CAR or CD19 polypeptide can consist of or comprise the amino acid sequence of SEQ ID NO:29 or 31 (mature CAR lacking a signal sequence); a useful CD19 CAR or CD19 polypeptide can consist of or comprise the amino acid sequence of SEQ ID NO:38 or 37 (mature CAR lacking a signal sequence); a useful CD19 CAR or CD19 polypeptide can consist of or comprise the amino acid sequence of SEQ ID NO:40 or 39 (mature CAR lacking a signal sequence); a useful CD19 CAR or CD19 polypeptide can consist of or comprise the amino acid sequence of SEQ ID NO:42 or 41 (mature CAR lacking a signal sequence); a useful CD19 CAR or CD19 polypeptide can consist of or comprise the amino acid sequence of SEQ ID NO:44 or 43 (mature CAR lacking a signal sequence); a useful CD19 CAR or CD19 polypeptide can consist of or comprise the amino acid sequence of SEQ ID NO:46 or 45 (mature CAR lacking a signal sequence); a useful CD19 CAR or CD19 polypeptide can consist of or comprise the amino acid sequence of SEQ ID NO:48 or 47 (mature CAR lacking a signal sequence). The CAR or polypeptide can be expressed in a form that includes a signal sequence, e.g., a human GM-CSF receptor alpha signal sequence (MLLLVTSLLLCELPHPAFLLIP; SEQ ID NO:36). The CAR or polypeptide can be expressed with additional sequences that are useful for monitoring expression, for example, a T2A skip sequence and a truncated EGFR. Thus, the CAR or polypeptide can comprise or consist of the amino acid sequence of SEQ ID Nos: 29, 30, 31, 33, 37, 38, 39, 40, 41, 42, 43, 44, 45, 46, 47, or 48 or can comprise or consist of an amino acid sequence that is at least 95%, 96%, 97%, 98% or 99% identical to SEQ ID NOs: 29, 30, 31, 33, 37, 38, 39, 40, 41, 42, 43, 44, 45, 46, 47, or 48. The CAR or polypeptide can comprise or consist of the amino acid sequence of any of SEQ ID NOs 1, 29, 30, 31, 32, 33, 37, 38, 39, 40, 41, 42, 43, 44, 45, 46, 47, or 48 with up to 1, 2, 3, 4 or 5 amino acid changes (preferably conservative amino acid changes).

In some embodiments, the nucleic acid encoding amino acid sequences SEQ ID NOs:1, 32, 29, 30, 31, 33, 37, 38, 39, 40, 41, 42, 43, 44, 45, 46, 47, and 48 are codon optimized.

Also disclosed is a polypeptide comprising a CD19 scFv comprising the amino acid sequence: DIQMTQSPSSLSASVGDRVTITCRASQDISKYLNWYQQKPGGAPKLLIYHTSRLHSGV PSRFSGSGSGTDFTFTISSLQQEDIATYYCQQGNTLPYTFGQGTKLEIKGSTSGSGKPG SGEGSTKGQVQLQESGPGLVAPSQTLSLTCTVSGVSLPDYGVSWIRQPPRKGLEWIGV IWGSETTYYNSALKSRVTISVDNSKNQFSLKLSSVTAADTAVYYCAKHYYYGGSYA MDYWGQGTLVTVSS (SEQ ID NO: 1) with up to 5 or up to 10 single amino acid substitutions.

Also disclosed is a polypeptide comprising a CD19 scFv comprising a light chain variable region that is at least 95% identical to: DIQMTQSPSSLSASVGDRVTITCRASQDISKYLNWYQQKPGGAPKLLIYHTSRLHSGV PSRFSGSGSGTDFTFTISSLQQEDIATYYCQQGNTLPYTFGQGTKLEIKGST (SEQ ID NO: 50) and a heavy chain variable region that is at least 95% identical to:

(SEQ ID NO: 54)
QVQLQESGPGLVAPSQTLSLTCTVSGVSLPDYGVSWIRQPPRKGLEWIGV
IWGSETTYYNSALKSRVTISVDNSKNQFSLKLSSVTAADTAVYYCAKHYY
YGGSYAMDYWGQGTLVTVSS.

In various cases, the CD19 scFv comprises a light chain variable region that comprises a CDR1 comprising: RASQDISKYLN (SEQ ID NO: 51), a CDR2 comprising HTSRLHS (SEQ ID NO: 52); and a CDR3 comprising QQGNTLPYT (SEQ ID NO: 53); and a heavy chain variable region that comprises a CDR1 comprising: DYGVS (SEQ ID NO: 57), a CDR2 comprising VIWGSETTYYNSALKS (SEQ ID NO: 60); and a CDR3 comprising HYYYGGSYAMDY (SEQ ID NO: 59).

Also disclosed is a polypeptide comprising a CD19 scFv comprising the amino acid sequence: DIQMTQSPSSLSASVGDRVTITCRASQDISKYLNWYQQKPGKAPKLLIYHTSRLHSGV PSRFSGSRSGTDFTLTISSLQPEDFATYYCQQGNTLPYTFGQGTKVEIKGSTSGSGKPG SGEGSTKGEVQLVESGGGLVQPGGSLRLSCAASGVSLPDYGVSWVRQAPGKGLEW VAVIWGSETTYYADSVKGRFTISADTSKNTAYLQMNSLRAEDTAVYYCSRHYYYGG SYAMDYWGQGTLVTVSS (SEQ ID NO:32) with up to 5 or up to 10 single amino acid substitutions.

Also disclosed is a polypeptide comprising a CD19 scFv comprising a light chain variable region that is at least 95% identical to: DIQMTQSPSSLSASVGDRVTITCRASQDISKYLNWYQQKPGKAPKLLIYHTSRLHSGV PSRFSGSRSGTDFTLTISSLQPEDFATYYCQQGNTLPYTFGQGTKVEIKGST (SEQ ID NO: 55) and a heavy chain variable region that is at least 95% identical to:

(SEQ ID NO: 56)
EVQLVESGGGLVQPGGSLRLSCAASGVSLPDYGVSWVRQAPGKGLEWVAV
IWGSETTYYADSVKGRFTISADTSKNTAYLQMNSLRAEDTAVYYCSRHYY
YGGSYAMDYWGQGTLVTVSS.

In various cases, the CD19 scFv comprises a light chain variable region that comprises a CDR1 comprising: RASQDISKYLN (SEQ ID NO: 51), a CDR2 comprising HTSRLHS (SEQ ID NO: 52); and a CDR3 comprising QQGNTLPYT (SEQ ID NO: 53) and a heavy chain variable region that comprises a CDR1 comprising: DYGVS (SEQ ID NO: 57), a CDR2 comprising VIWGSETTYYADSVKG (SEQ ID NO: 58); and a CDR3 comprising HYYYGGSYAMDY (SEQ ID NO: 59).

Also disclosed is a chimeric antigen receptor comprising a CD19 scFv; a spacer; a transmembrane domain; a co-stimulatory domain; and a CD3ζ signaling domain, wherein the CD19 scFv is selected from the group consisting of: (a) an ScFv comprising the amino acid sequence DIQMTQSPSSLSASVGDRVTITCRASQDISKYLNWYQQKPGGAPKLLIYHTSRLHSGV PSRFSGSGSGTDFTFTISSLQQEDIATYYCQQGNTLPYTFGQGTKLEIKGSTSGSGKPG SGEGSTKGQVQLQESGPGLVAPSQTLSLTCTVSGVSLPDYGVSWIRQPPRKGLEWIGV IWGSETTYYNSALKSRVTISVDNSKNQFSLKLSSVTAADTAVYYCAKHYYYGGSYA MDYWGQGTLVTVSS (SEQ ID NO: 1) with up to 5 or up to 10 single amino acid substitutions; (b) an scFv comprising a light chain variable region that is at least 95% identical to: DIQMTQSPSSLSASVGDRVTITCRASQDISKYLNWYQQKPGGAPKLLIYHTSRLHSGV PSRFSGSGSGTDFTFTISSLQQEDIATYYCQQGNTLPYTFGQGTKLEIKGST (SEQ ID NO: 50) and a heavy chain variable region that is at least 95% identical to: QVQLQESGPGLVAPSQTLSLTCTVSGVSLPDYGVSWIRQPPRKGLEWIGVIWGSETT YYNSALKSRVTISVDNSKNQFSLKLSSVTAADTAVYYCAKHYYYGGSYAMDYWGQ GTLVTVSS (SEQ ID NO: 54); (c) an scFv comprising a light chain variable region that comprises a CDR1 comprising: RASQDISKYLN (SEQ ID NO: 51), a CDR2 comprising HTSRLHS (SEQ ID NO: 52); and a CDR3 comprising QQGNTLPYT (SEQ ID NO: 53); and a heavy chain variable region that comprises a CDR1 comprising: DYGVS (SEQ ID NO: 57), a CDR2 comprising VIWGSETTYYNSALKS (SEQ ID NO: 60); and a CDR3 comprising HYYYGGSYAMDY (SEQ ID NO: 59); (d) an scFv comprising the amino acid sequence: DIQMTQSPSSLSASVGDRVTITCRASQDISKYLNWYQQKPGKAPKLLIYHTSRLHSGV PSRFSGSRSGTDFTLTISSLQPEDFATYYCQQGNTLPYTFGQGTKVEIKGSTSGSGKPG SGEGSTKGEVQLVESGGGLVQPGGSLRLSCAASGVSLPDYGVSWVRQAPGKGLEW VAVIWGSETTYYADSVKGRFTISADTSKNTAYLQMNSLRAEDTAVYYCSRHYYYGG SYAMDYWGQGTLVTVSS (SEQ ID NO:32) with up to 5 or up to 10 single amino acid substitutions; (e) an scFv comprising a light chain variable region that is at least 95% identical to: DIQMTQSPSSLSASVGDRVTITCRASQDISKYLNWYQQKPGKAPKLLIYHTSRLHSGV PSRFSGSRSGTDFTLTISSLQPEDFATYYCQQGNTLPYTFGQGTKVEIKGST (SEQ ID NO: 55) and a heavy chain variable region that is at least 95% identical to: EVQLVESGGGLVQPGGSLRLSCAASGVSLPDYGVSWVRQAPGKGLEWVAVIWGSE TTYYADSVKGRFTISADTSKNTAYLQMNSLRAEDTAVYYCSRHYYYGGSYAMDYW GQGTLVTVSS (SEQ ID NO: 56); and (f) a scFv comprising a light chain variable region that comprises a CDR1 comprising: RASQDISKYLN (SEQ ID NO: 51), a CDR2 comprising HTSRLHS (SEQ ID NO: 52); and a CDR3 comprising QQGNTLPYT (SEQ ID NO: 53) and a heavy chain variable region that comprises a CDR1 comprising: DYGVS (SEQ ID NO: 57), a CDR2 comprising VIWGSETTYYADSVKG (SEQ ID NO: 58); and a CDR3 comprising HYYYGGSYAMDY (SEQ ID NO: 59).

In various embodiments of the chimeric antigen receptor of claim 8, wherein the transmembrane domain is selected from: a CD4 transmembrane domain, a CD8 transmembrane domain, or a CD28 transmembrane domain; the transmembrane domain is a CD28 transmembrane domain; chimeric antigen receptor of claim 8, wherein the costimulatory domain is a CD28, 4-1BB, OX40 or a 2B4 costimulatory domain; the costimulatory domain comprises the amino acid sequence of any of SEQ ID NOs:22-25 and 49; the CD3ζ signaling domain comprises the amino acid sequence of SEQ ID NO:21; a linker of 3 to 15 amino acids is located between the costimulatory domain and the CD3ζ signaling domain; and the spacer comprises any one of SEQ ID NOs:2-12.

### Spacer Region

The CAR or polypeptide described herein can include a spacer located between the CD19 targeting domain (i.e., a CD19 targeted ScFv or variant thereof) and the transmembrane domain. A variety of different spacers can be used. Some of them include at least portion of a human Fc region, for example a hinge portion of a human Fc region or a CH3 domain or variants thereof. Table 1 below provides various spacers that can be used in the CARs described herein.

TABLE 1
Examples of Spacers

| Name | Length | Sequence |
|---|---|---|
| a3 | 3 aa | AAA |
| linker | 10 aa | GGGSSGGGSG (SEQ ID NO: 2) |
| IgG4 hinge (S→P) | 12 aa | ESKYGPPCPPCP (SEQ ID NO: 3) |
| (S228P) | | |
| IgG4 hinge | 12 aa | ESKYGPPCPSCP (SEQ ID NO: 4) |
| IgG4 hinge (S228P) + linker | 22 aa | ESKYGPPCPPCPGGGSSGGGSG (SEQ ID NO: 5) |
| CD28 hinge | 39 aa | IEVMYPPPYLDNEKSNGTIIHVKGKHLCPSPLFPGPSKP (SEQ ID NO: 6) |
| CD8 hinge-48 aa | 48 aa | AKPTTTPAPRPPTPAPTIASQPLSLRPEACRPAAGGAVHTRGLDFACD (SEQ |
| | | ID NO: 7) |
| CD8 hinge-45 aa | 45 aa | TTTPAPRPPTPAPTIASQPLSLRPEACRPAAGGAVHTRGLDFACD (SEQ ID |
| | | NO: 8) |
| IgG4(HL-CH3) | 129 aa | ESKYGPPCPPCPGGGSSGGGSGGQPREPQVYTLPPSQEEMTKNQVSLTCL |
| Also called IgG4(HL-ΔCH2) | | VKGFYPSDIAVEWESNGQPENNYKTTPPVLDSDGSFFLYSRLTVDKSRWQE |
| (includes S228P in hinge) | | GNVFSCSVMHEALHNHYTQKSLSLSLGK (SEQ ID NO: 9) |
| IgG4(L235E, N297Q) | 229 aa | ESKYGPPCPSCPAPEFEGGPSVFLFPPKPKDTLMISRTPEVTCVVVDVSQED |
| | | PEVQFNWYVDGVEVHNAKTKPREEQFQSTYRVVSVLTVLHQDWLNGKEY |
| | | KCKVSNKGLPSSIEKTISKAKGQPREPQVYTLPPSQEEMTKNQVSLTCLVKG |
| | | FYPSDIAVEWESNGQPENNYKTTPPVLDSDGSFFLYSRLTVDKSRWQEGN |
| | | VFSCSVMHEALHNHYTQKSLSLSLGK (SEQ ID NO: 10) |
| IgG4(S228P, L235E, N297Q) | 229 aa | ESKYGPPCPPCPAPEFEGGPSVFLFPPKPKDTLMISRTPEVTCVVVDVSQED |
| | | PEVQFNWYVDGVEVHNAKTKPREEQFQSTYRVVSVLTVLHQDWLNGKEY |
| | | KCKVSNKGLPSSIEKTISKAKGQPREPQVYTLPPSQEEMTKNQVSLTCLVKG |
| | | FYPSDIAVEWESNGQPENNYKTTPPVLDSDGSFFLYSRLTVDKSRWQEGN |
| | | VFSCSVMHEALHNHYTQKSLSLSLGK (SEQ ID NO: 11) |
| IgG4(CH3) | 107 aa | GQPREPQVYTLPPSQEEMTKNQVSLTCLVKGFYPSDIAVEWESNGQPENN |
| Also called IgG4(ΔCH2) | | YKTTPPVLDSDGSFFLYSRLTVDKSRWQEGNVFSCSVMHEALHNHYTQKSL |
| | | SLSLGK (SEQ ID NO: 12) |

Some spacer regions include all or part of an immunoglobulin (e.g., IgG1, IgG2, IgG3, IgG4) hinge region, i.e., the sequence that falls between the CH1 and CH2 domains of an immunoglobulin, e.g., an IgG4 Fc hinge or a CD8 hinge. Some spacer regions include an immunoglobulin CH3 domain (called CH3 or ΔCH2) or both a CH3 domain and a CH2 domain. The immunoglobulin derived sequences can include one or more amino acid modifications, for example, 1, 2, 3, 4 or 5 substitutions, e.g., substitutions that reduce off-target binding.

The hinge/linker region can also comprise an IgG4 hinge region having the sequence ESKYGPPCPSCP (SEQ ID NO:4) or ESKYGPPCPPCP (SEQ ID NO:3). The hinge/linger region can also comprise the sequence ESKYGPPCPPCP (SEQ ID NO:3) followed by the linker sequence GGGSSGGGSG (SEQ ID NO:2) followed by IgG4 CH3 sequence GQPREPQVYTLPPSQEEMTKNQVSLTCLVKGFYPSDIAVEWESNGQPENNYKTTPPV LDSDGSFFLYSRLTVDKSRWQEGNVFSCSVMHEALHNHYTQKSLSLSLGK (SEQ ID NO: 12). Thus, the entire linker/spacer region can comprise the sequence: ESKYGPPCPPCPGGGSSGGGSGGQPREPQVYTLPPSQEEMTKNQVSLTCLVKGFYPS DIAVEWESNGQPENNYKTTPPVLDSDGSFFLYSRLTVDKSRWQEGNVFSCSVMHEA LHNHYTQKSLSLSLGK (SEQ ID NO:11). In some cases, the spacer has 1, 2, 3, 4, or 5 single amino acid changes (e.g., conservative changes) compared to SEQ ID NO: 11. In some cases, the IgG4 Fc hinge/linker region that is mutated at two positions (L235E; N297Q) in a manner that reduces binding by Fc receptors (FcRs).

### Transmembrane Domain

A variety of transmembrane domains can be used in the. Table 2 includes examples of suitable transmembrane domains. Where a spacer region is present, the transmembrane domain (TM) is located carboxy terminal to the spacer region.

TABLE 2
Examples of Transmembrane Domains

| Name | Accession | Length | Sequence |
|---|---|---|---|
| CD3z | J04132.1 | 21 aa | LCYLLDGILFIYGVILTALFL (SEQ ID NO: 13) |
| CD28 | NM_006139 | 27 aa | FWVLVVVGGVLACYSLLVTVAFIIFWV (SEQ ID NO: 14) |
| CD28(M) | NM_006139 | 28 aa | MFWVLVVVGGVLACYSLLVTVAFIIFWV (SEQ ID NO: 15) |
| CD4 | M35160 | 22 aa | MALIVLGGVAGLLLFIGLGIFF (SEQ ID NO: 16) |
| CD8tm | NM_001768 | 21 aa | IYIWAPLAGTCGVLLLSLVIT (SEQ ID NO: 17) |
| CD8tm2 | NM_001768 | 23 aa | IYIWAPLAGTCGVLLLSLVITLY (SEQ ID NO: 18) |
| CD8tm3 | NM_001768 | 24 aa | IYIWAPLAGTCGVLLLSLVITLYC (SEQ ID NO: 19) |
| 41BB | NM_001561 | 27 aa | IISFFLALTSTALLFLLFF LTLRFSVV (SEQ ID NO: 20) |
| NKG2D | NM_007360 | 21 aa | PFFFCCFIAVAMGIRFIIMVA (SEQ ID NO: 61) |

### Costimulatory Domain

The costimulatory domain can be any domain that is suitable for use with a CD3ζ signaling domain. In some cases the co-signaling domain is a 4-1BB co-signaling domain that includes a sequence that is at least 90%, at least 95%, at least 98% identical to or identical to: KRGRKKLLYIFKQPFMRPVQTTQEEDGCSCRFPEEEEGGCEL (SEQ ID NO:24). In some cases, the 4-1BB co-signaling domain has 1, 2, 3, 4 of 5 amino acid changes (preferably conservative) compared to SEQ ID NO:24.

The costimulatory domain(s) are located between the transmembrane domain and the CD3ζ signaling domain. Table 3 includes examples of suitable costimulatory domains together with the sequence of the CD3ζ signaling domain.

TABLE 3
CD3ζ Domain and Examples of Costimulatory Domains

| Name | Accession | Length | Sequence |
|---|---|---|---|
| CD3ζ | J04132.1 | 113 aa | RVKFSRSADAPAYQQGQNQLYNELNLGRREEYDVLDKRRGR |
| | | | DPEMGGKPRRKNPQEGLYNELQKDKMAEAYSEIGMKGERR |
| | | | RGKGHDGLYQGLSTATKDTYDALHMQALPPR (SEQ ID |
| | | | NO: 21) |
| CD28 | NM_006139 | 42 aa | RSKRSRLLHSDYMNMTPRRPGPTRKHYQPYAPPRDFAAYRS |
| | | | (SEQ ID NO: 22) |
| CD28gg* | NM_006139 | 42 aa | RSKRSRGGHSDYMNMTPRRPGPTRKHYQPYAPPRDFAAYR |
| | | | S (SEQ ID NO: 23) |
| 41BB | NM_001561 | 42 aa | KRGRKKLLYIFKQPFMRPVQTTQEEDGCSCRFPEEEEGGCEL |
| | | | (SEQ ID NO: 24) |
| OX40 | NM_003327 | 42 aa | ALYLLRRDQRLPPDAHKPPGGGSFRTPIQEEQADAHSTLAKI |
| | | | (SEQ ID NO: 25) |
| 2B4 | NM_016382 | 120 aa | WRRKRKEKQSETSPKEFLTIYEDVKDLKTRRNHEQEQTFPGG |
| | | | GSTIYSMIQSQSSAPTSQEPAYTLYSLIQPSRKSGSRKRNHSPS |
| | | | FNSTIYEVIGKSQPKAQNPARLSRKELENFDVYS (SEQ ID |
| | | | NO: 49) |

In various embodiments: the costimulatory domain is selected from the group consisting of a costimulatory domain depicted in Table 3 or a variant thereof having 1-5 (e.g., 1 or 2) amino acid modifications, a CD28 costimulatory domain or a variant thereof having 1-5 (e.g., 1 or 2) amino acid modifications, a 4-1BB costimulatory domain or a variant thereof having 1-5 (e.g., 1 or 2) amino acid modifications and an OX40 costimulatory domain or a variant thereof having 1-5 (e.g., 1 or 2) amino acid modifications. In certain embodiments, a 4-1BB costimulatory domain or a variant thereof having 1-5 (e.g., 1 or 2) amino acid modifications in present. In some embodiments there are two costimulatory domains, for example a CD28 co-stimulatory domain or a variant thereof having 1-5 (e.g., 1 or 2) amino acid modifications (e.g., substitutions) and a 4-1BB co-stimulatory domain or a variant thereof having 1-5 (e.g., 1 or 2) amino acid modifications (e.g., substitutions). In various embodiments the 1-5 (e.g., 1 or 2) amino acid modification are substitutions. The costimulatory domain is amino terminal to the CD3ζ signaling domain and a short linker consisting of 2-10, e.g., 3 amino acids (e.g., GGG) is can be positioned between the costimulatory domain and the CD3ζ signaling domain.

### CD3ζ Signaling Domain

The CD3ζ Signaling domain can be any domain that is suitable for use with a CD3ζ signaling domain. In some cases, the CD3ζ signaling domain includes a sequence that is at least 90%, at least 95%, at least 98% identical to or identical to: RVKFSRSADAPAYQQGQNQLYNELNLGRREEYDVLDKRRGRDPEMGGKPRRKNPQ EGLYNELQKDKMAEAYSEIGMKGERRRGKGHDGLYQGLSTATKDTYDALHMQAL PPR (SEQ ID NO:21). In some cases, the CD3 signaling has 1, 2, 3, 4 of 5 amino acid changes (preferably conservative) compared to SEQ ID NO:21.

### Truncated EGFR or CD19

The CD3ζ signaling domain can be followed by a ribosomal skip sequence (e.g., LEGGGEGRGSLLTCGDVEENPGPR; SEQ ID NO:27) and a truncated EGFR having a sequence that is at least 90%, at least 95%, at least 98% identical to or identical to: LVTSLLLCELPHPAFLLIPRKVCNGIGIGEFKDSLSINATNIKHFKNCTSISGDLHILPVA FRGDSFTHTPPLDPQELDILKTVKEITGFLLIQAWPENRTDLHAFENLEIIRGRTKQHG QFSLAVVSLNITSLGLRSLKEISDGDVIISGNKNLCYANTINWKKLFGTSGQKTKIISN RGENSCKATGQVCHALCSPEGCWGPEPRDCVSCRNVSRGRECVDKCNLLEGEPREF VENSECIQCHPECLPQAMNITCTGRGPDNCIQCAHYIDGPHCVKTCPAGVMGENNTL VWKYADAGHVCHLCHPNCTYGCTGPGLEGCPTNGPKIPSIATGMVGALLLLLVVAL GIGLFM (SEQ ID NO:28). In some cases, the truncated EGFR has 1, 2, 3, 4 of 5 amino acid changes (preferably conservative) compared to SEQ ID NO:28. Alternatively the CD3ζ signaling domain can be followed by a ribosomal skip sequence (e.g., LEGGGEGRGSLLTCGDVEENPGPR; SEQ ID NO:27) and a truncated CD19R (also called CD19t) having a sequence that is at least 90%, at least 95%, at least 98% identical to or identical to:

(SEQ ID NO: 26)
MPPPRLLFFLLFLTPMEVRPEEPLVVKVEEGDNAVLQCLKGTSDGPTQQL
TWSRESPLKPFLKLSLGLPGLGIHMRPLAIWLFIFNVSQQMGGFYLCQPG
PPSEKAWQPGWTVNVEGSGELFRWNVSDLGGLGCGLKNRSSEGPSSPSGK
LMSPKLYVWAKDRPEIWEGEPPCVPPRDSLNQSLSQDLTMAPGSTLWLSC
GVPPDSVSRGPLSWTHVHPKGPKSLLSLELKDDRPARDMWVMETGLLLPR
ATAQDAGKYYCHRGNLTMSFHLEITARPVLWHWLLRTGGWKVSAVTLAYL
IFCLCSLVGILHLQRALVLRRKR

An amino acid modification refers to an amino acid substitution, insertion, and/or deletion in a protein or peptide sequence. An “amino acid substitution” or “substitution” refers to replacement of an amino acid at a particular position in a parent peptide or protein sequence with another amino acid. A substitution can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. The following are examples of various groupings of amino acids: 1) Amino acids with nonpolar R groups: Alanine, Valine, Leucine, Isoleucine, Proline, Phenylalanine, Tryptophan, Methionine; 2) Amino acids with uncharged polar R groups: Glycine, Serine, Threonine, Cysteine, Tyrosine, Asparagine, Glutamine; 3) Amino acids with charged polar R groups (negatively charged at pH 6.0): Aspartic acid, Glutamic acid; 4) Basic amino acids (positively charged at pH 6.0): Lysine, Arginine, Histidine (at pH 6.0). Another grouping may be those amino acids with phenyl groups: Phenylalanine, Tryptophan, and Tyrosine.

In some cases, the CD19 CAR or CD19 polypeptide can be produced using a vector in which the CAR open reading frame is followed by a T2A ribosome skip sequence and a truncated EGFR (EGFRt), which lacks the cytoplasmic signaling tail. In this arrangement, co-expression of EGFRt provides an inert, non-immunogenic surface marker that allows for accurate measurement of gene modified cells, and enables positive selection of gene-modified cells, as well as efficient cell tracking of the therapeutic T cells in vivo following adoptive transfer. Efficiently controlling proliferation to avoid cytokine storm and off-target toxicity is an important hurdle for the success of T cell immunotherapy. The EGFRt incorporated in the CD19 CAR lentiviral vector can act as suicide gene to ablate the CAR+ T cells in cases of treatment-related toxicity.

The CAR or polypeptide described herein can be produced by any means known in the art, though preferably it is produced using recombinant DNA techniques. Nucleic acids encoding the several regions of the chimeric receptor can be prepared and assembled into a complete coding sequence by standard techniques of molecular cloning known in the art (genomic library screening, overlapping PCR, primer-assisted ligation, site-directed mutagenesis, etc.) as is convenient. The resulting coding region is preferably inserted into an expression vector and used to transform a suitable expression host cell line, preferably a T lymphocyte, and most preferably an autologous T lymphocyte.

Various T cell subsets isolated from the patient can be transduced with a vector for CAR or polypeptide expression. Central memory T cells are one useful T cell subset. Central memory T cell can be isolated from peripheral blood mononuclear cells (PBMC) by selecting for CD45RO+/CD62L+ cells, using, for example, the CliniMACS® device to immunomagnetically select cells expressing the desired receptors. The cells enriched for central memory T cells can be activated with anti-CD3/CD28, transduced with, for example, a lentiviral vector that directs the expression of an CD19 CAR or as well as a non-immunogenic surface marker for in vivo detection, ablation, and potential ex vivo selection. The activated/genetically modified CD19 central memory T cells can be expanded in vitro with IL-2/IL-15 and then cryopreserved. Additional methods of preparing CART cells can be found in PCT/US2016/043392.

Methods for preparing useful T cell populations are described in, for example, WO 2017/015490 and WO 2018/102761. In some cases, it may be useful to use natural killer (NK) cells, e.g., allogenic NK cells derived from peripheral blood or cord blood. In other cases, NK cells can be derived from human embryonic stem cells (hESCs) or induced pluripotent stem cells (iPSCs).

In some embodiments, described herein is a composition comprising the iPSC-derived CAR T cells or CAR NK cells. In some embodiments, a composition comprising iPSC-derived CAR T cells or CAR NK cells has enhanced therapeutic properties. In some embodiments, the iPSC-derived CAR T cells or CAR NK cells demonstrate enhanced functional activity including potent cytokine production, cytotoxicity and cytostatic inhibition of tumor growth, e.g. as activity that reduces the amount of tumor load.

The CAR can be transiently expressed in a T cell population by an mRNA encoding the CAR. The mRNA can be introduced into the T cells by electroporation (Wiesinger et al. 2019 Cancers (Basel) 11:1198).

In some embodiments, a composition comprising the CAR T cells comprise one or more of helper T cells, cytotoxic T cells, memory T cells, naïve T cells, regulatory T cells, natural killer T cells, or combinations thereof.

In some embodiments, described herein is a method of increasing survival of a subject having cancer comprising administering a composition comprising a CAR T cell or CAR NK cell described herein.

In some embodiments, described herein is a method of treating a cancer in a patient comprising administering a composition comprising a CAR T cell or CAR NK cell described herein.

In some embodiments, described herein is a method of reducing or ameliorating a symptom associated with a cancer in a patient comprising administering a composition comprising a CAR T cell or CAR NK cell described herein.

In some embodiments, a composition comprising CAR T cells or CAR NK cells described herein is administered locally or systemically. In some embodiments, a composition comprising CAR T cells or CAR NK cells described herein is administered by single or repeat dosing.

The materials, methods, and examples are illustrative only and not intended to be limiting. All publications, patent applications, patents, sequences, database entries, and other references mentioned herein are incorporated by reference in their entirety for any and all purposes.

Other features and advantages of the described compositions and methods will be apparent from the following detailed description and figures, and from the claims.

### DESCRIPTION OF DRAWINGS

FIG. 1. Affinity titration of anti-CD19 scFvs. The parental murine scFv, FMC63 (squares) and the two humanized variants, VH4Vκ1 (circles) and 4D5 (triangles), were displayed on the surface of yeast. The affinities of the indicated scFvs were determined by flow cytometry with titration of biotinylated CD19 and fit to a 1:1 equilibrium binding model. Affinities are presented as mean±standard deviation of 3 trials. Titration curve fits are calculated based on the average K_{d} for each clone. (B) The thermal stabilities of the indicated scFvs were determined by heating yeast displaying the indicated scFvs followed by labeling with either 50 nM biotinylated CD19 (FMC63) or conformation-specific binder biotinylated protein L (humanized variants). Fluorescence proportional to the degree of foldedness was determined by flow cytometry. Thermal stabilities are presented as mean±standard deviation of 3-4 trials. The Tₘ for each clone is represented by a diamond. Curves are fit to splines.

FIG. 2. Thermal stability of anti-CD19 scFvs. The parental murine scFv, FMC63 (squares), and the two humanized variants, VH4Vκ1 (circles) and 4D5 (triangles), were displayed on the surface of yeast. The thermal stabilities of the indicated scFvs were determined by heating yeast displaying the indicated scFvs to the noted temperatures followed by labeling with either 50 nM biotinylated CD19 (FMC63) or biotinylated protein L (humanized variants). Fluorescence proportional to the degree of foldedness of the scFvs was determined by flow cytometry. Thermal stabilities are presented as mean±standard deviation of 3-4 trials. Curves are fit to a two-state unfolding model using the calculated average Tₘ for each clone.

FIG. 3A-C. CAR design and T cell engineering. (A) Diagram of lentiviral cassette design for CD19-targeted CARs including the EF1α promoter, GM-CSF signal peptide sequence, CD19 scFv, IgG4 hinge and CH3 connected by a 10 amino acid GS linker (ch2Δ), CD4 transmembrane domain, cytoplasmic 4-1BB costimulatory domain and cytoplasmic CD3ζ stimulatory domain. A non-signaling truncated EGFR is separated from the CAR by a T2A ribosome skip sequence for tracking of transduction efficiency. (B) Pictorial representation of the CD19 CAR. (C) CD19 CARs were expressed in T_{N/MEM} by lentiviral transduction. T cells were labeled either with anti-EGFR (left) or anti-Fc (right) to assess CAR expression. Representative histograms are shown.

FIG. 4A-C. Characterization of CAR T cell killing in vitro. T_{N/MEM} expressing CAR that included FMC63 (squares), VH4Vκ1 (circles) or 4D5 (triangles) were analyzed for their ability to kill CD19_{low} KG1a (A), CD19+ Daudi (B), and CD19+ SUP-B15 (C) at the specified effector:target ratios over the course of 3 days. Percentage killing is relative to mock and is presented as mean of two independent wells in one experiment.

FIG. 5A-C. Characterization of CAR T cell killing in vitro. T_{N/MEM} expressing the CAR that included FMC63 (squares), VH4Vκ1 (circles) or 4D5 (triangles) were analyzed for their ability to kill CD19_{low} KG1a (A), CD19+ Daudi (B), and CD19+ SUP-B15 (C) at the specified effector:target ratios over the course of 3 days. Percentage killing is relative to mock and is presented as mean of two independent wells in one experiment.

FIG. 6A-B. T cell expansion during tumor rechallenge. T_{N/MEM} expressing CAR that included FMC63 (squares), VH4Vκ1 (circles) or 4D5 (triangles) were challenged with CD19+ Raji cells at the specified effector:target (E:T) ratio on day 0. CAR T cells were subsequently rechallenged with the same number of Raji cells every 3 days. CAR T cell counts were assessed by flow cytometry. Fold expansions are relative to the number of T cells plated on day 0. Data are presented as mean±standard deviation of four independent experiments using T cells from two different healthy donors.

FIG. 7A-B. T cells harboring humanized CD19-targeted CARs demonstrate therapeutic efficacy in NALM6 model. 1×10⁶ ffluc+ NALM6 cells were administered intravenously to NSG mice. Five days post engraftment, 1×10⁶ mock transduced or CAR-expressing T_{N/MEM} were administered intravenously. (A) NALM6 tumor growth was monitored by luminescence imaging. Dotted lines denote luminescence of individual mice. Solid lines denote average luminescence for each group. (B) Kaplan-Meier survival analysis for each group (n=10 mice).

FIG. 8A-B. T cells harboring humanized CD19-targeted CARs demonstrate therapeutic efficacy in Raji model. 0.5×10⁶ ffluc+ Raji cells were administered intravenously to NSG mice. Five days post engraftment, 1×10⁶ mock transduced or CAR-expressing T_{N/MEM} were administered intravenously. (A) Raji tumor growth was monitored by luminescence imaging. Dotted lines denote luminescence of individual mice. Solid lines denote average luminescence for each group. (B) Kaplan-Meier survival analysis for each group (n=10 mice).

FIG. 9A-D. Production of a cell line with low density CD19 expression. Raji KO cells were transduced with low CD19 expressing plasmid pGK100 and low density cell suspension was cloned (yellow) by limiting dilution (A). CD19 molecules on Raji Low cells (B) and primary acute leukemic cells (C) are presented. The combined bar graph of (B) and (C) is shown in (D).

FIG. 10A-D. CD19 CAR with VH4Vκ1 show anti-tumor efficacy in an in vivo low CD19 expression Raji model. Cloned and propagated low CD19 expressing Raji cells (ffluc+) (0.5×10⁶) were inoculated into NSG. Mice were treated with 1×10⁶ CAR T cells (huCD19-CAR T cells (4D5 or VH4Vκ1) or murine CD19-CAR T cells (FMC63) with the 41BB costimulatory domain) 7 days later. Tumor burden was followed by weekly bioluminescent imaging (A). Survival curves were generated (B). Blood samples were collected at euthanasia and human T cells and CAR T cells present in mouse blood were analyzed by flow cytometry (C and D).

FIG. 11A-C. HuCD19-CAR (VH4Vκ1) show anti-tumor efficacy in an in vivo CD19+ tumor model. (A) Immune-compromised mice bearing CD19-expressing Raji tumors were treated with huCD19-CAR T cells 4D5 or VH4Vκ1, or murine CD19-CAR T cells containing different costimulatory domains (41BB or CD28). Tumor growth was monitored by weekly bioluminescent imaging (B) and survival curves were generated (C).

FIG. 12A-B. HuCD19-CAR T cells with VH4Vκ1 scFv produce IFNγ and degranulation against Raji cells that express low and high levels of CD19. The humanized CD19 scFvs 4D5 and VH4Vκ1 CAR T cells were co-cultured with CD19+ Raji cells at 1:1 ratio in the form of CD19 low and high (parental) for 12 hours and intracellular IFNγ were analyzed with flow cytometry. CD19 KO Raji cells were used as negative control.

FIG. 13A-H. Amino acid sequence of VH4Vκ1-IgG4 (HL-ΔCH2)-CD4TM-41BB (A; SEQ ID NO:31, 4D5-IgG4 (HL-ΔCH2)-CD4TM-41BB (B; SEQ ID NO:33), VH4Vκ1-IgG4 (ΔCH2)-CD4TM-41BB (C; SEQ ID NO:37), 4D5-IgG4(ΔCH2)-CD4TM-41BB (D; SEQ ID NO:39), VH4Vκ1-IgG4 (HL-ΔCH2)-CD8TM-41BB (E; SEQ ID NO:41), 4D5-IgG4 (HL-ΔCH2)-CD8TM-41BB (F; SEQ ID NO:43), VH4Vκ1-IgG4(ΔCH2)-CD8TM-41BB (G; SEQ ID NO:45), and 4D5-IgG4(ΔCH2)-CD8TM-41BB (H; SEQ ID NO:47).

### DETAILED DESCRIPTION

In this disclosure, CD19 scFv and the generation and anti-tumor efficacy of CAR with an anti-CD19 scFv antigen-binding domain are described, inter alia.

### EXAMPLES

The CD19 svFvs, CAR that include the scFv and their use is further described in the following examples, which do not limit the scope the claims.

### Materials and Methods

### Cell Lines

The cell lines (Raji and NALM6) were cultured in RPMI-1640 (Lonza) containing 10% fetal bovine serum (FBS, Hyclone) (complete RPMI). All cells were cultured at 37° C. with 5% CO₂. HUT78 cells were cultured in IMDM (Iscove's Modified Dulbecco's Medium; Fisher Scientific) with 20% FBS.

### DNA Constructs and Lentivirus Production

Tumor cells were engineered to express enhanced green fluorescent protein and firefly luciferase (eGFP/ffluc) by transduction with epHIV7 lentivirus carrying the eGFP/ffluc fusion under the control of the EF1α promoter as described previously (Lenalidomide Enhances the Function of CS1 Chimeric Antigen Receptor-Redirected T Cells Against Multiple Myeloma (Wang et al). Clinical Cancer Research 2018).

Research grade lentivirus was generated using a modified polyethylenimine (PEI) mediated transfection method (Optimization of lentiviral vector production using polyethylenimine-mediated transfection. Yong Tang, et al. Oncology Letters. 2015). Briefly, 293T cells were transfected with packaging plasmid and CAR lentiviral backbone plasmid using a modified PEI method. Viral supernatants were collected after 3 to 4 days. Supernatants were concentrated via high-speed centrifugation and lentiviral pellets were resuspended in phosphate-buffered saline (PBS)-lactose solution (4 g lactose per 100 mL PBS), aliquoted and stored at −80° C. Lentiviral titers were quantified using HT1080 cells based on EGFRt expression.

### Cell Isolation, CAR Lentiviral Transduction, and Ex Vivo Expansion

Leukapheresis products were obtained from consented research participants (healthy donors) under protocols approved by the City of Hope Internal Review Board (IRB). On the day of leukapheresis, peripheral blood mononuclear cells (PBMC) were isolated by density gradient centrifugation over Ficoll-Paque (GE Healthcare) followed by multiple washes in PBS/EDTA (Miltenyi Biotec). Cells were rested overnight at room temperature (RT) on a rotator, and subsequently washed and resuspended in X-VIVO T cell medium (Lonza) containing 10% FBS (complete X-VIVO). Up to 5.0×10⁹ PBMC were incubated with anti-CD14 and anti-CD25 microbeads (Miltenyi Biotec) for 30 min at RT and magnetically depleted using the CliniMACS® system (Miltenyi Biotec) according to the manufacturer's protocol and these were termed depleted PBMCs (dPBMC), dPBMC were frozen in CryoStor® CS5 (StemCell Technologies) until further processing. Tn/mem cells were prepared from dPBMC by staining with anti-CD62L microbeads (Miltenyi Biotec) and enriching CD62L+ cells using AutoMACS system, dPBMC or Tn/mem were stimulated with CD3/CD28 Dyna-beads (Thermal Fisher Scientific, Ratio of Cell to Beads is 1 to 2) in X-vivo15 medium with 10 U/mL IL2 and 0.5 ng/mL IL15.

Lentiviral transduction was performed. Briefly T cells were cultured with CD3/CD28 Dynabeads® (Life Technologies), protamine sulfate (APP Pharmaceuticals), cytokine mixture (as stated above) and desired lentivirus at a multiplicity of infection (MOI) of 1-3. Cells were then cultured in and replenished with fresh complete X-VIVO containing cytokines every 2-3 days. After 7 days, beads were magnetically removed, and cells were further expanded in complete X-VIVO containing cytokines to achieve desired cell yield. Following further expansion, cells were frozen in CryoStor® CS5 prior to in vitro functional assays and in vivo tumor models. Purity and phenotype of CAR T cells were verified by flow cytometry.

### Flow Cytometry

T cells were harvested and stained as described previously (Jonnalagadda, M., et al., Chimeric antigen receptors with mutated IgG4 Fc spacer avoid fc receptor binding and improve T cell persistence and antitumor efficacy. Mol Ther, 2015. 23(4):757-68). T cell phenotype was examined using fluorochrome-conjugated antibodies against CD3, CD4, CD8α, CD45 (clone H130, BC-8 or 94.1). Transgenic CAR expression was determined by staining of the truncated EGFR tag. Data were acquired on MacsQuant Analyzer 10 (Miltenyi Biotec) flow cytometers and analyzed with FlowJo (v10.6.1).

The affinities of the scFvs were determined by flow cytometry with titration of biotinylated CD19 and fit to a 1:1 equilibrium binding model.

The thermal stabilities of the scFvs were determined by heating yeast displaying the indicated scFvs followed by labeling with either 50 nM biotinylated CD19 (FMC63) or conformation-specific binder biotinylated protein L (humanized variants VH4Vk1 and 4D5). Fluorescence proportional to the degree of foldedness was determined by flow cytometry.

### In Vitro T Cell Assays

For tumor killing assays, CAR T cells and tumor targets were co-cultured at indicated effector:tumor (E:T) ratios. To test cytotoxicity effect of the CAR T cells, GFP expressing tumor cells were plated in 96-well U-bottom plates at the indicated density. Effector cells (CAR T or Mock T cells) were washed, resuspended in fresh medium without cytokines and co-cultured with the indicated tumor cells for the specified number of hours. Cytotoxicity was routinely evaluated by flow cytometry with enumeration of GFP+DAPI− tumor cells for viable GFP-expressing tumor cells.

To test for degranulation activity, CAR T or control T cells were incubated with tumor cells for five hours in the presence of CD107a antibody and GolgiStop protein transport inhibitor (BD Biosciences). After the co-culture, cells were harvested, fixed, permeabilized, and stained for intracellular cytokines. Degranulation (CD107a staining) and intracellular cytokine staining (e.g. IFNγ) were examined by flow cytometry.

### In Vivo Tumor Studies

All animal experiments were performed under protocols approved by the City of Hope Institutional Animal Care and Use Committee. Tumor xenograft models were generated using 6 to 8 week-old NOD/SCID/IL2R−/− (NSG) mice as previously described (Jackson Laboratory) [Urak, R., et al., Ex vivo Akt inhibition promotes the generation of potent CD19CAR T cells for adoptive immunotherapy. J Immunother Cancer, 2017. 5:26]. Briefly, 1×10⁶ ffluc+ NALM6 cells, 0.5×10⁶ low CD19 expressing Raji cells (ffluc+), or 0.5×10⁶ ffluc+CD19-expressing Raji cells were injected intravenously (i.v.) into the NSG mice. After a specified number of days (e.g., 5 or 7 days), mice were then treated with CAR T cells or mock T cells as described for each experiment. Tumor growth was determined by in vivo bio-photonic imaging using a Xenogen IVIS 100. Mice were also monitored for survival, with euthanasia applied according to the American Veterinary Medical Association Guidelines.

### Example 1: Affinity and Thermal Stability of Humanized CD19 scFv

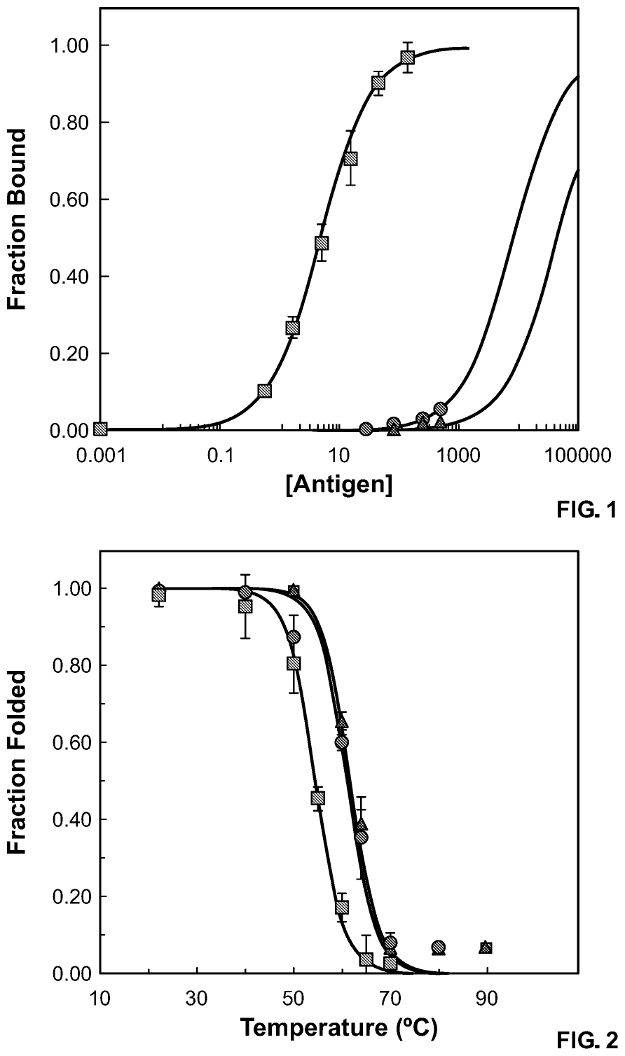

The parental murine scFv, FMC63, and the two humanized variants, VH4Vκ1 and 4D5, were displayed on the surface of yeast. The affinities of the scFvs were determined by flow cytometry with titration of biotinylated CD19 and fit to a 1:1 equilibrium binding model (FIG. 1). The thermal stabilities of the scFvs were determined by heating yeast displaying the indicated scFvs followed by labeling with either 50 nM biotinylated CD19 (FMC63) or conformation-specific binder biotinylated protein L (humanized variants). Fluorescence proportional to the degree of foldedness was determined by flow cytometry.

The affinity of FMC63 was 4.8±0.7 nM. The affinity of VH4Vk1 was 8,300±1,000. The affinity of 4DK was 45,000±30,000.

The parental murine scFv, FMC63, and the two humanized variants, VH4Vκ1 and 4D5, were displayed on the surface of yeast. The thermal stabilities of the indicated scFvs were determined by heating yeast displaying the indicated scFvs to the noted temperatures followed by labeling with either 50 nM biotinylated CD19 (FMC63) or biotinylated protein L (humanized variants) (FIG. 2). Fluorescence proportional to the degree of foldedness of the scFvs was determined by flow cytometry. The thermal stability of FMC63 was 55±0.7° C. The thermal stability of VH4Vk1 was 61±0.9° C. The thermal stability of 4DK was 62±0.6° C.

### Example 2: In Vitro Activity of VH4Vk1 CAR T Cells and 4D5 CAR T Cells

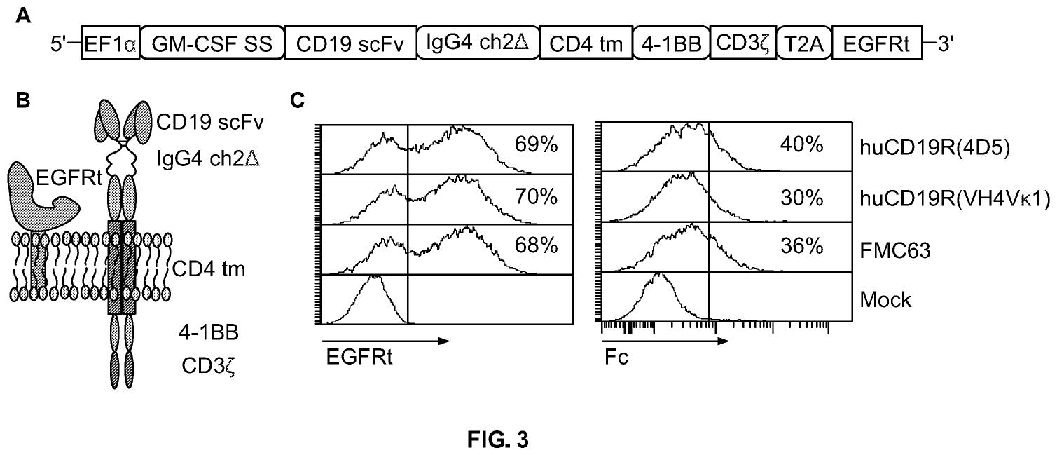

Each of the humanized scFv and FMC63 scFv was incorporated into a CAR that included an IgG4(ΔCH2) spacer (SEQ ID NO: 12), a CD4 transmembrane domain (SEQ ID NO: 16), a 4-1BB co-stimulatory domain (SEQ ID NO: 24), and a CD3zeta domain (SEQ ID NO: 21). The CAR was a preceded by a GM-CSF signal sequence and was followed by a T2A skip sequence and a truncated EGFR (FIG. 3A). The T2A skip sequence and the truncated EGFR permit co-expression of a membrane bound truncated EGFR that can act as a marker for CAR expression (FIG. 3B). The CD19 CAR were expressed by lentivirus in TN/MEM cells. The T cells were labelled with anti-EGFR antibody (FIG. 3C, left) or anti-Fc antibody (FIG. 3C, right).

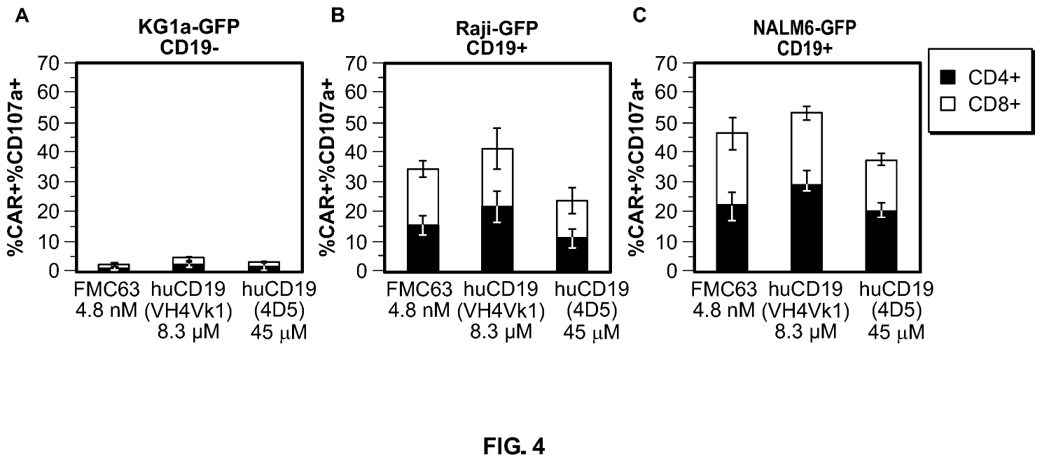

To assess the activity of the CAR in vitro, TN/MEM expressing each of the constructs were tested for their ability to degranulate when exposed with CD19_{low} KG1a cells, CD19+ Raji cells, or CD19+ NALM6 cells. The results of this analysis are presented in FIG. 4A-C.

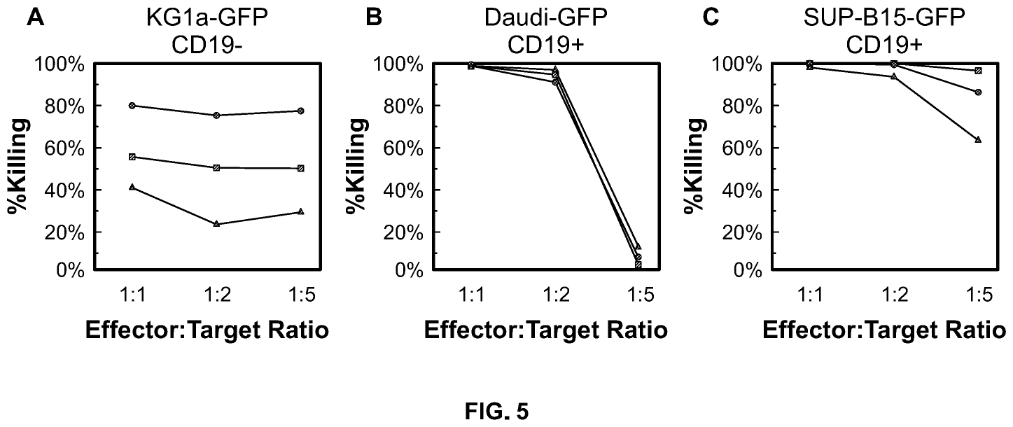

T_{N/MEM} expressing each of the CAR were assessed for the ability to kill CD19_{low} KG1a cells, CD19+ Daudi cells, and CD19+ SUP-B15 cells at an Effector:Target ratio of 1:1, 1:2 or 1:5. The results of this analysis are presented in FIG. 5A-C.

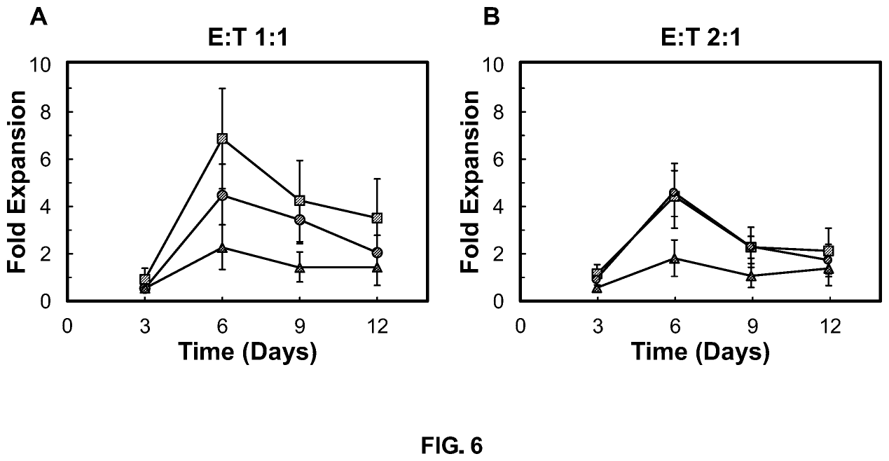

Expansion of CAR T cells during rechallenge was assessed. Briefly, CAR constructs incorporating FMC63, VH4Vk1 or 4D5 were challenged with CD19+ Raji cells at an effector:target (E:T) ratio of 1:1 (FIG. 6A) or 2:1 (FIG. 6B) on day 0. CAR T cells were subsequently rechallenged with the same number of Raji cells every 3 days. CAR T cell counts were assessed by flow cytometry.

### Example 3: In Vivo Activity of VH4Vk1 CAR T Cells and 4D5 CAR T Cells

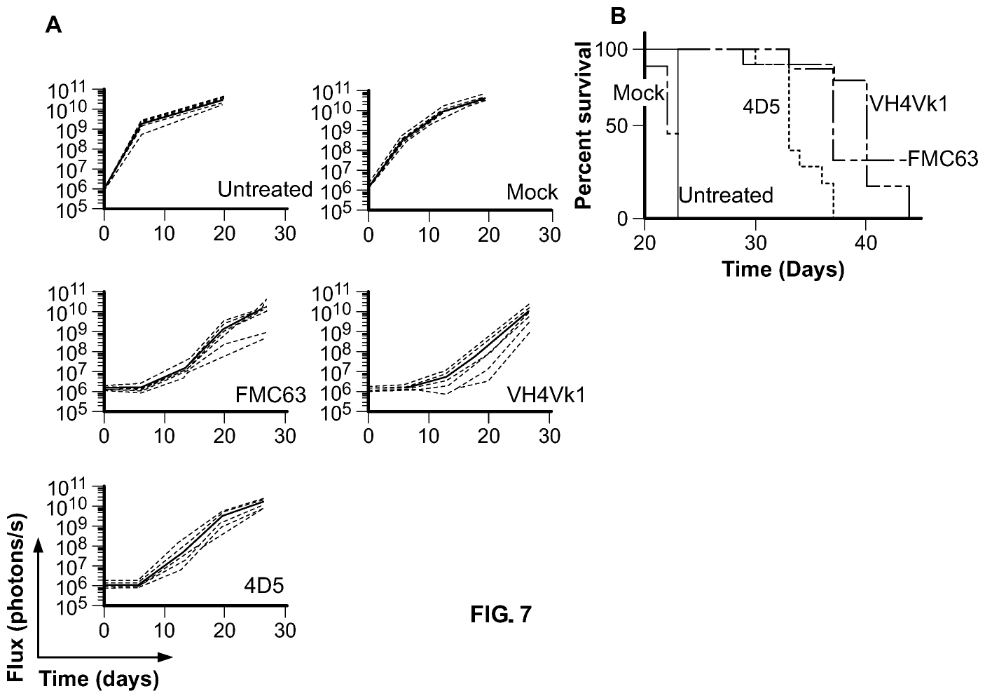

The in vivo activity of FMC63 CAR T cells, VH4Vk1 CAR T cells and 4D5 CAR T cells was assessed in a NALM6 model. Briefly. 1×10⁶ ffluc+ NALM6 cells were administered intravenously to NSG mice. Five days post engraftment, 1×10⁶ mock transduced or CAR-expressing T_{N/MEM} were administered intravenously. The results of this analysis are presented in FIG. 7A-B.

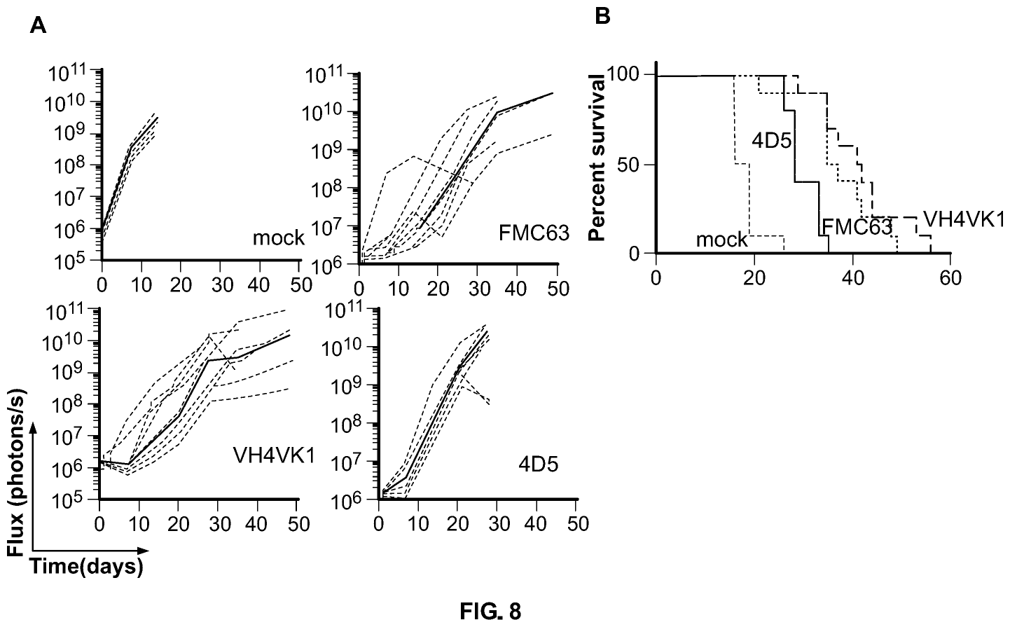

The in vivo activity of FMC63 CAR T cells, VH4Vk1 CAR T cells and 4D5 CAR T cells was assessed in a Raji model. Briefly. 1×10⁶ ffluc+ NALM6 cells were administered intravenously to NSG mice. Five days post engraftment, 1×10⁶ mock transduced or CAR-expressing T_{N/MEM} were administered intravenously. The results of this analysis are presented in FIG. 8A-B.

### Example 4: VH4Vk1 CAR T Cells and 4D5 CAR T Cells can Target Cells with Low CD19 Expression

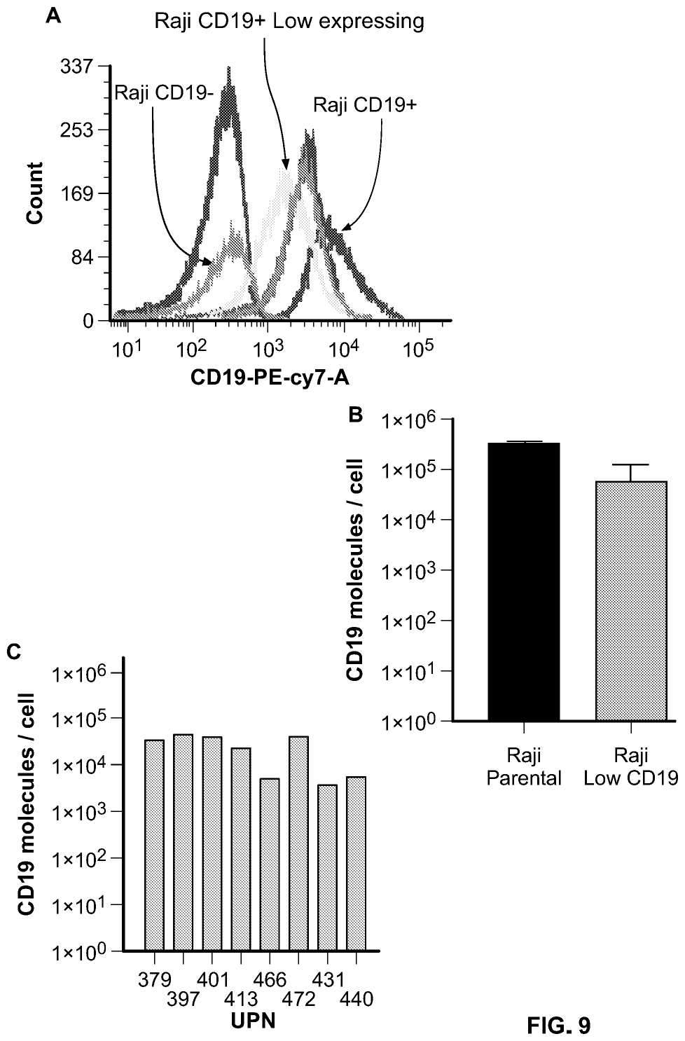

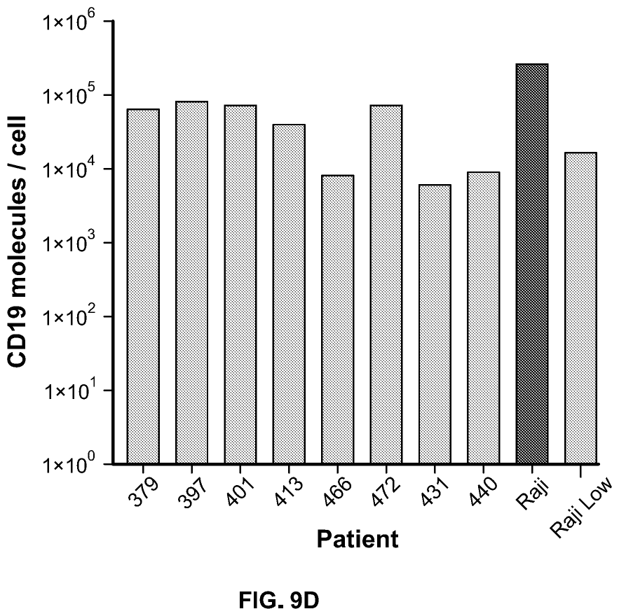

CD19 knockout Raji cells were transduced with low CD19 expressing plasmid pGK100 and low CD19 density cell cells were cloned by limiting dilution (FIGS. 9A and 9B). Leukemic cells isolated from patients with ALL were analyzed for CD19 molecule/cell with flow cytometry. CD19 expression levels on parental Raji (high expresser) and derivative Raji low (CD19 low expresser) were used as controls. As shown in FIGS. 9C and 9D, CD19 expression on the Raji CD19 Low cells was similar to that on various primary acute leukemic cell lines.

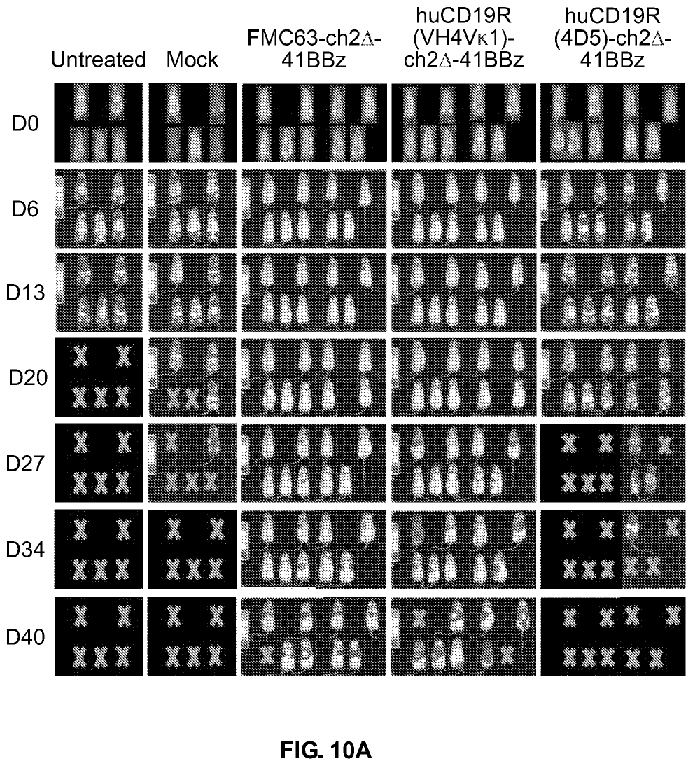

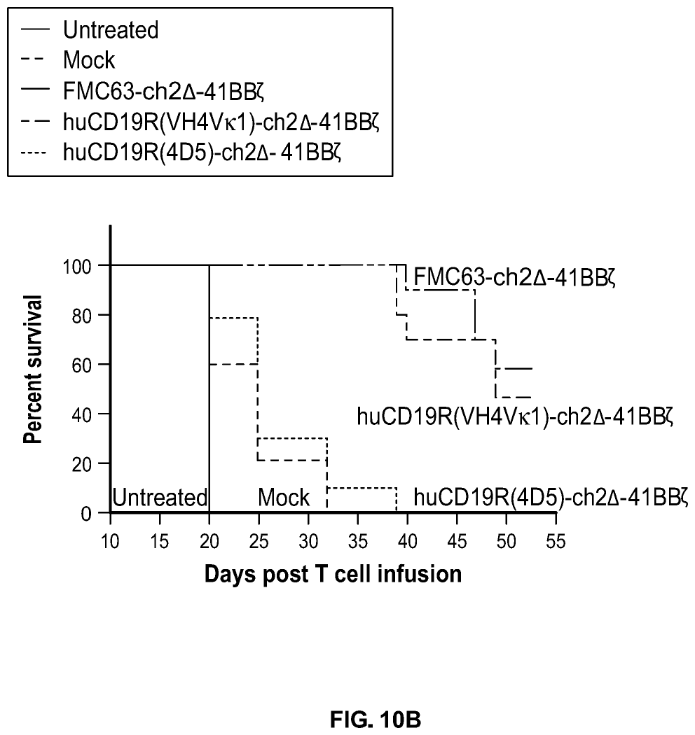

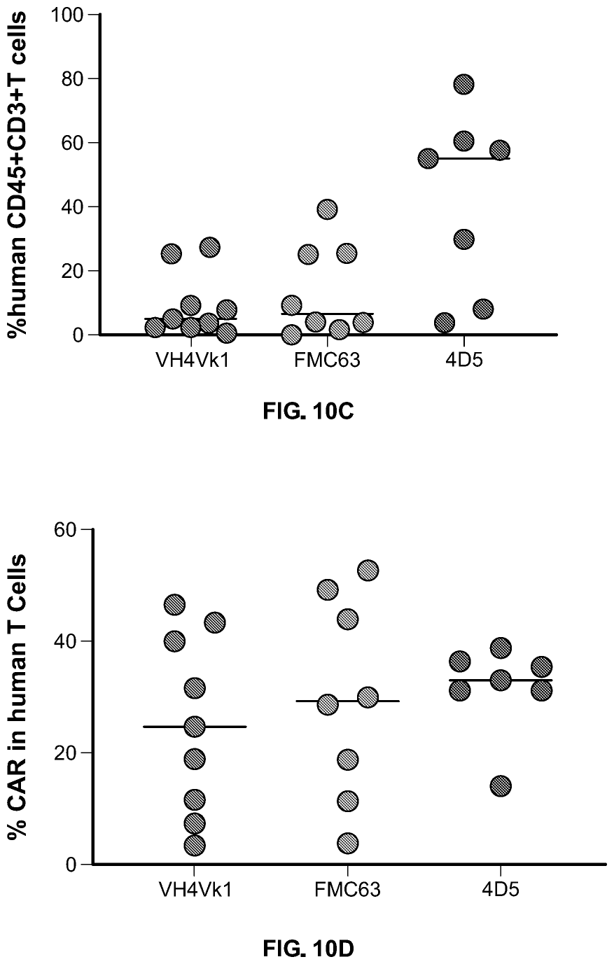

Cloned and propagated low CD19 expressing Raji cells (ffluc+) (0.5×10⁶) were inoculated into NSG. Mice were treated with 1×10⁶ CAR T cells 7 days later. Tumor burden was followed by weekly bioluminescent imaging (FIG. 10A). The mice bearing CD19-low Raji tumor cells were treated with huCD19-CAR T cells (4D5 or VH4Vκ1) or murine CD19-CAR T cells (FMC63) with the 41BB costimulatory domain. Survival curves were generated, and HuCD19-CAR T cells with VH4Vκ1 scFv exhibited superior anti-tumor activity and prolonged mouse survival (FIG. 10B). The survival data for treating the low CD19-expressing tumors with human VH4Vκ1 CD19-CAR was comparable to treating with the murine FMC63 CD19-CAR. Blood samples were collected at euthanasia, and the presence of human T cells and CAR T cells present in mouse blood were analyzed (FIGS. 10C-10D).

### Example 5. Human CD19-CAR (VH4Vκ1 and 4D5) Show Anti-Tumor Efficacy in an In Vivo CD19+ Tumor Model

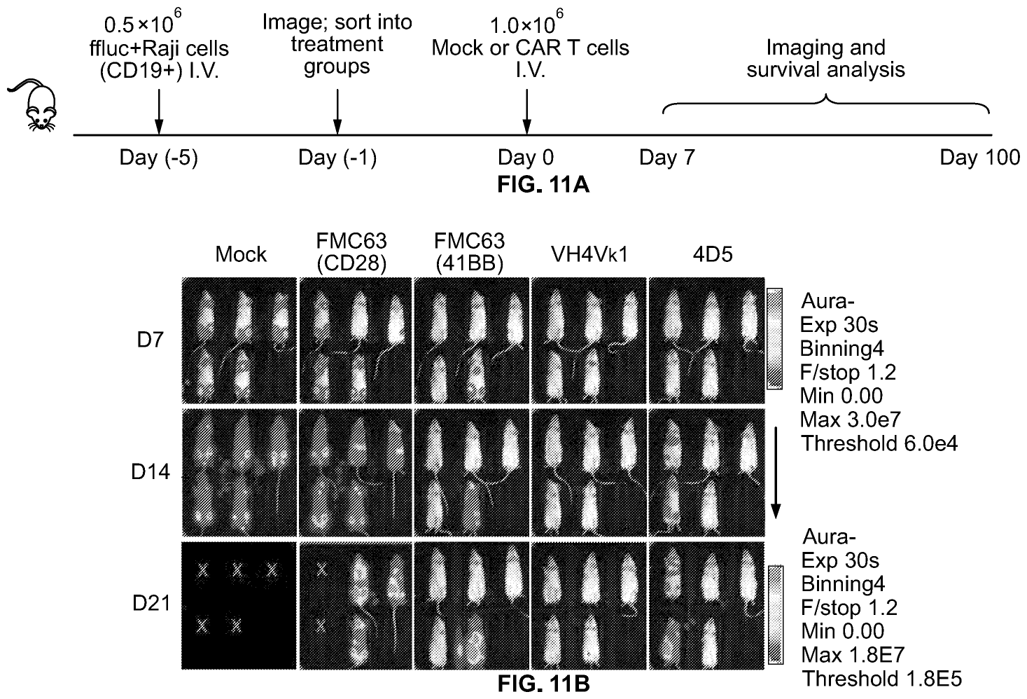

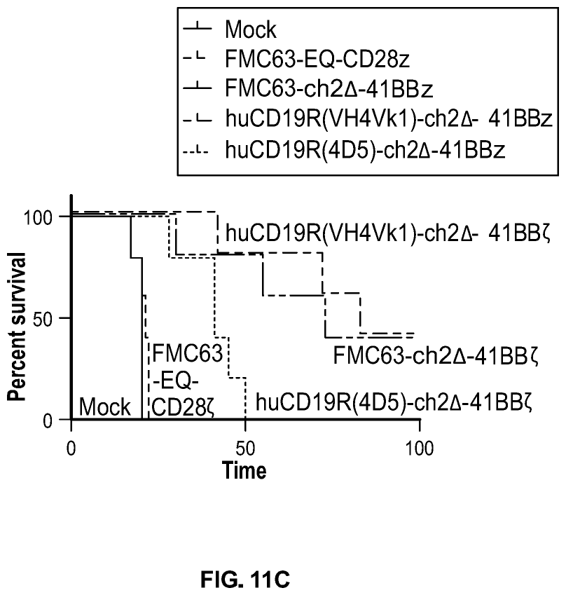

NSG mice bearing CD19-expressing Raji tumors were treated with 1×10⁶ huCD19-CAR T cells (4D5 or VH4Vκ1), or murine CD19-CAR T cells (FMC63) containing different costimulatory domains (FIG. 11A). Tumor growth was monitored by weekly bioluminescent imaging and survival curves were generated (FIGS. 11B and 11C). HuCD19-CAR T cells with VH4Vκ1 scFv exhibited superior anti-tumor activity and prolonged mouse survival. The survival data for treating the CD19-expressing tumors with human VH4Vκ1 CD19-CAR was comparable to treating with the murine FMC63 CD19-CAR containing 41BB.

### Example 6. HuCD19-CAR T Cells with VH4Vκ1 scFv Produce IFNγ and Degranulation Against Raji Cells that Express Low and High Levels of CD19

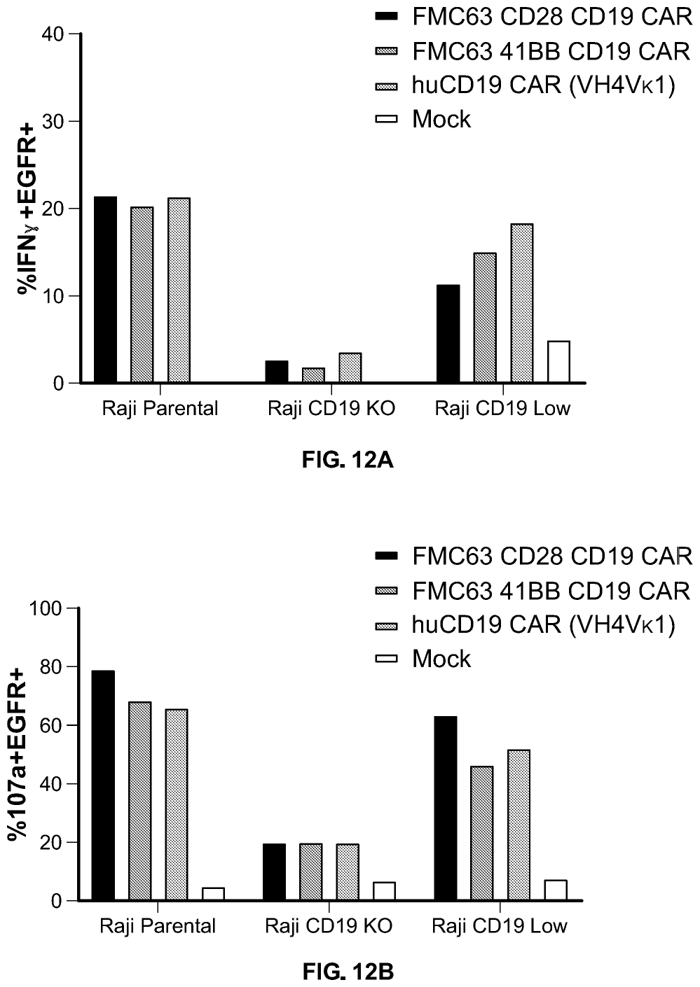

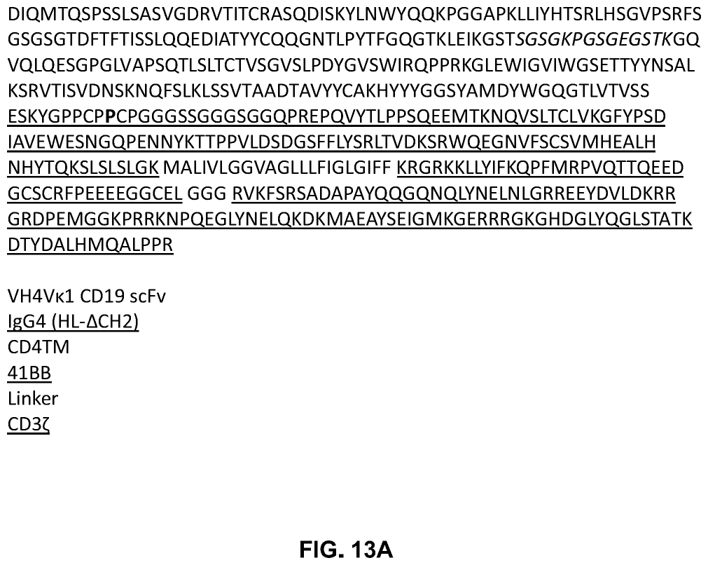

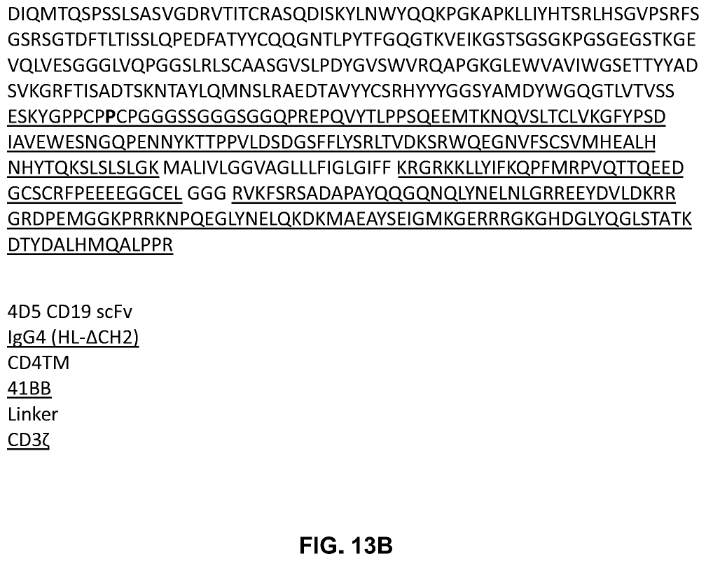

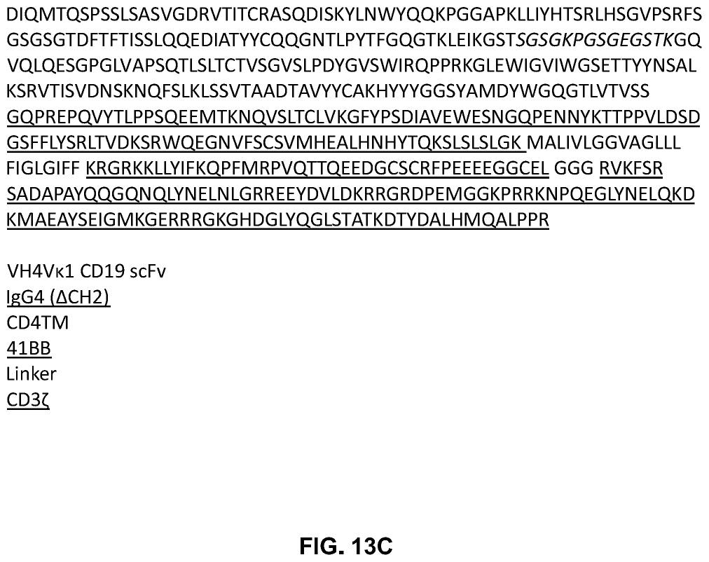

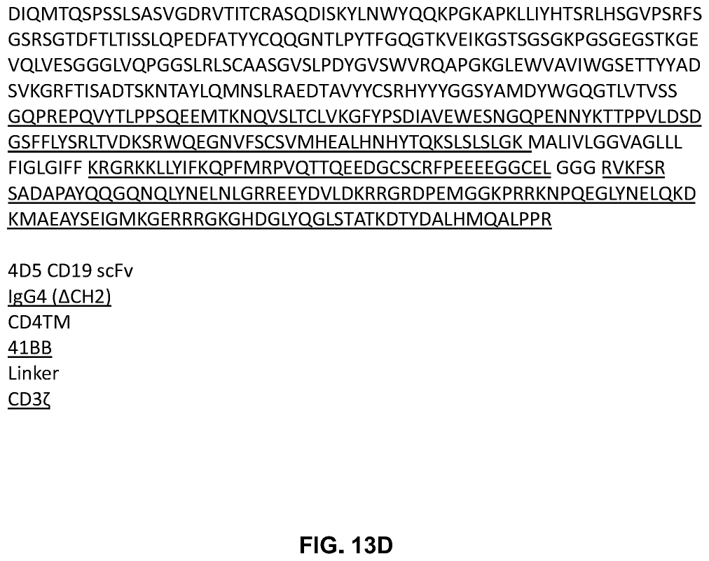

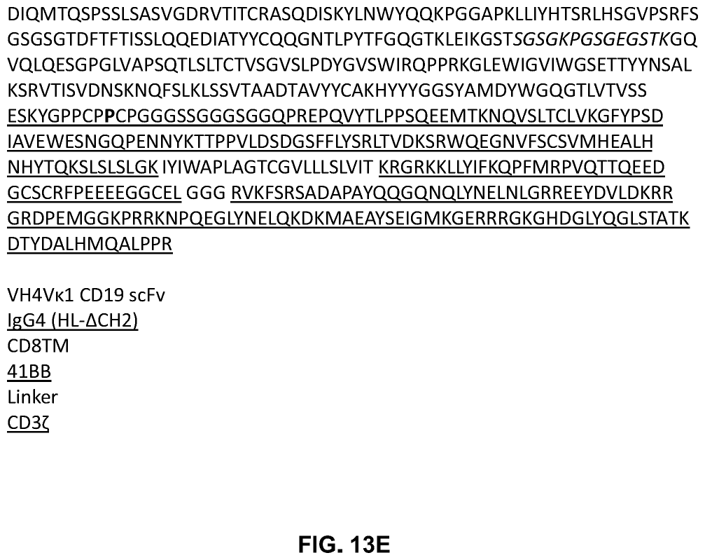

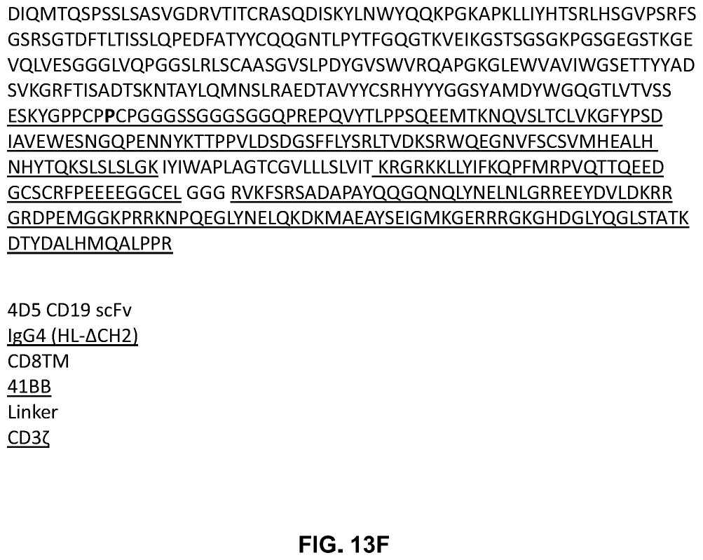

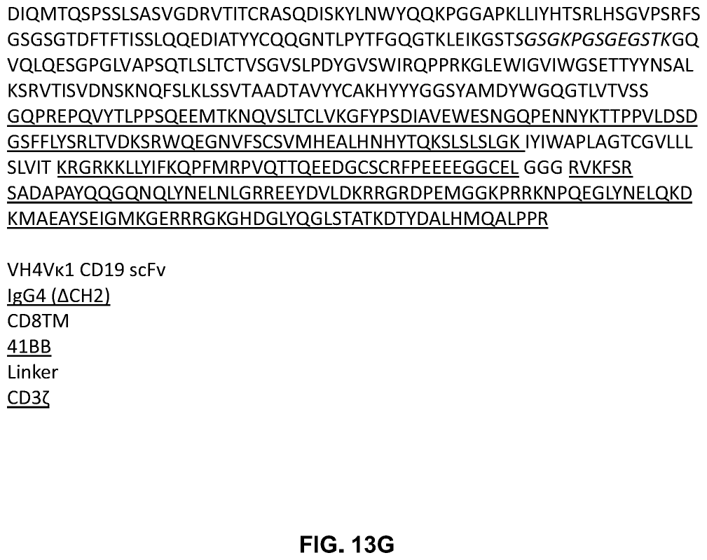

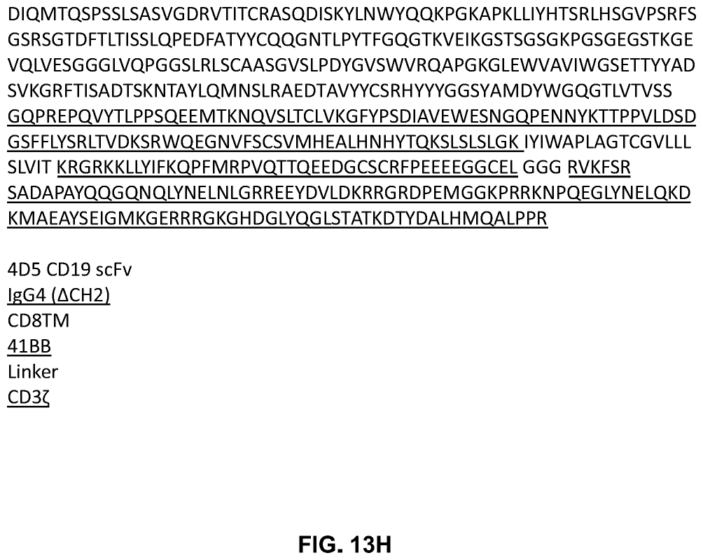

The humanized CD19 scFv VH4Vκ1 CAR T cells were co-cultured with CD19+ Raji cells (CD19 low and high (parental)) at 1:1 ratio for 12 hours. CD19 KO Raji cells were used as negative control. Intracellular IFNγ were analyzed with flow cytometry (FIG. 12A). Degranulation was also measured (FIG. 12B). The VH4Vκ1 humanized CD19 CAR exhibited efficient anti-tumor effector function, comparable to murine version FMC63 (CD19 41BB CAR). Data with CAR T cells from different donors are presented.

### OTHER EMBODIMENTS

It is to be understood that while the invention has been described in conjunction with the detailed description thereof, the foregoing description is intended to illustrate and not limit the scope of the invention.

1. A polypeptide comprising a CD19 scFv comprising: (a) the amino acid sequence:
DIQMTQSPSSLSASVGDRVTITCRASQDISKYLNWYQQKPGGAPKLLIYHTSRLHSGVPS RFSGSGSGTDFTFTISSLQQEDIATYYCQQGNTLPYTFGQGTKLEIKGSTSGSGKPGSGEG STKGQVQLQESGPGLVAPSQTLSLTCTVSGVSLPDYGVSWIRQPPRKGLEWIGVIWGSET TYYNSALKSRVTISVDNSKNQFSLKLSSVTAADTAVYYCAKHYYYGGSYAMDYWGQG TLVTVSS (SEQ ID NO: 1) with up to 5 or up to 10 single amino acid substitutions; or
(b) a light chain variable region that is at least 95% identical to:
(SEQ ID NO: 50)
DIQMTQSPSSLSASVGDRVTITCRASQDISKYLNWYQQKPGGAPKLLIYH
TSRLHSGVPSRFSGSGSGTDFTFTISSLQQEDIATYYCQQGNTLPYTFGQ
GTKLEIKGST
and a heavy chain variable region that is at least 95% identical to:
(SEQ ID NO: 54)
QVQLQESGPGLVAPSQTLSLTCTVSGVSLPDYGVSWIRQPPRKGLEWIGV
IWGSETTYYNSALKSRVTISVDNSKNQFSLKLSSVTAADTAVYYCAKHYY
YGGSYAMDYWGQGTLVTVSS.

2. (canceled)

3. The polypeptide of claim 1, wherein the CD19 scFv comprises a light chain variable region that comprises a CDR1 comprising: RASQDISKYLN (SEQ ID NO: 51), a CDR2 comprising HTSRLHS (SEQ ID NO: 52); and a CDR3 comprising QQGNTLPYT (SEQ ID NO: 53); and a heavy chain variable region that comprises a CDR1 comprising: DYGVS (SEQ ID NO: 57), a CDR2 comprising VIWGSETTYYNSALKS (SEQ ID NO: 60); and a CDR3 comprising HYYYGGSYAMDY (SEQ ID NO: 59).

4. A polypeptide comprising a CD19 scFv comprising (a) the amino acid sequence:
DIQMTQSPSSLSASVGDRVTITCRASQDISKYLNWYQQKPGKAPKLLIYHTSRLHSGVPS RFSGSRSGTDFTLTISSLQPEDFATYYCQQGNTLPYTFGQGTKVEIKGSTSGSGKPGSGEG STKGEVQLVESGGGLVQPGGSLRLSCAASGVSLPDYGVSWVRQAPGKGLEWVAVIWG SETTYYADSVKGRFTISADT SKNTAYLQMNSLRAEDTAVYYCSRHYYYGGSYAMDYW GQGTLVTVSS (SEQ ID NO:32) with up to 5 or up to 10 single amino acid substitutions; or
(b) a light chain variable region that is at least 95% identical to: DIQMTQSPSSLSASVGDRVTITCRASQDISKYLNWYQQKPGKAPKLLIYHTSRLHSGVPS RFSGSRSGTDFTLTISSLQPEDFATYYCQQGNTLPYTFGQGTKVEIKGST (SEQ ID NO: 55 and a heavy chain variable region that is at least 95% identical to:
(SEQ ID NO: 56)
EVQLVESGGGLVQPGGSLRLSCAASGVSLPDYGVSWVRQAPGKGLEWVAV
IWGSETTYYADSVKGRFTISADTSKNTAYLQMNSLRAEDTAVYYCSRHYY
YGGSYAMDYWGQGTLVTVSS.

5. (canceled)

6. The polypeptide of claim 4, wherein the CD19 scFv comprises a light chain variable region that comprises a CDR1 comprising: RASQDISKYLN (SEQ ID NO: 51), a CDR2 comprising HTSRLHS (SEQ ID NO: 52); and a CDR3 comprising QQGNTLPYT (SEQ ID NO: 53) and a heavy chain variable region that comprises a CDR1 comprising: DYGVS (SEQ ID NO: 57), a CDR2 comprising VIWGSETTYYADSVKG (SEQ ID NO: 58); and a CDR3 comprising HYYYGGSYAMDY (SEQ ID NO: 59).

7. A nucleic acid molecule comprising a sequence encoding the polypeptide of claim 1.

8. A chimeric antigen receptor comprising a CD19 scFv; a spacer; a transmembrane domain; a co-stimulatory domain; and a CD3ζ signaling domain, wherein the CD19 scFv is selected from the group consisting of:
(a) an scFv comprising the amino acid sequence DIQMTQSPSSLSASVGDRVTITCRASQDISKYLNWYQQKPGGAPKLLIYHTSRLHSGVPS RFSGSGSGTDFTFTISSLQQEDIATYYCQQGNTLPYTFGQGTKLEIKGSTSGSGKPGSGEG STKGQVQLQESGPGLVAPSQTLSLTCTVSGVSLPDYGVSWIRQPPRKGLEWIGVIWGSET TYYNSALKSRVTISVDNSKNQFSLKLSSVTAADTAVYYCAKHYYYGGSYAMDYWGQG TLVTVSS (SEQ ID NO: 1) with up to 5 or up to 10 single amino acid substitutions;
(b) an scFv comprising a light chain variable region that is at least 95% identical to:
(SEQ ID NO: 50)
DIQMTQSPSSLSASVGDRVTITCRASQDISKYLNWYQQKPGGAPKLLIYH
TSRLHSGVPSRFSGSGSGTDFTFTISSLQQEDIATYYCQQGNTLPYTFGQ
GTKLEIKGST
and a heavy chain variable region that is at least 95% identical to:
(SEQ ID NO: 54)
QVQLQESGPGLVAPSQTLSLTCTVSGVSLPDYGVSWIRQPPRKGLEWIGV
IWGSETTYYNSALKSRVTISVDNSKNQFSLKLSSVTAADTAVYYCAKHYY
YGGSYAMDYWGQGTLVTVSS;
(c) an scFv comprising a light chain variable region that comprises a CDR1 comprising: RASQDISKYLN (SEQ ID NO: 51), a CDR2 comprising HTSRLHS (SEQ ID NO: 52); and a CDR3 comprising QQGNTLPYT (SEQ ID NO: 53); and a heavy chain variable region that comprises a CDR1 comprising: DYGVS (SEQ ID NO: 57), a CDR2 comprising VIWGSETTYYNSALKS (SEQ ID NO: 60); and a CDR3 comprising HYYYGGSYAMDY (SEQ ID NO: 59);
(d) an scFv comprising the amino acid sequence: DIQMTQSPSSLSASVGDRVTITCRASQDISKYLNWYQQKPGKAPKLLIYHTSRLHSGVPS RFSGSRSGTDFTLTISSLQPEDFATYYCQQGNTLPYTFGQGTKVEIKGSTSGSGKPGSGEG STKGEVQLVESGGGLVQPGGSLRLSCAASGVSLPDYGVSWVRQAPGKGLEWVAVIWG SETTYYADSVKGRFTISADTSKNTAYLQMNSLRAEDTAVYYCSRHYYYGGSYAMDYW GQGTLVTVSS (SEQ ID NO:32) with up to 5 or up to 10 single amino acid substitutions;
(e) an scFv comprising a light chain variable region that is at least 95% identical to: DIQMTQSPSSLSASVGDRVTITCRASQDISKYLNWYQQKPGKAPKLLIYHTSRLHSGVPS RFSGSRSGTDFTLTISSLQPEDFATYYCQQGNTLPYTFGQGTKVEIKGST (SEQ ID NO: 55) and a heavy chain variable region that is at least 95% identical to: EVQLVESGGGLVQPGGSLRLSCAASGVSLPDYGVSWVRQAPGKGLEWVAVIWGSETTY YADSVKGRFTISADTSKNTAYLQMNSLRAEDTAVYYCSRHYYYGGSYAMDYWGQGTL VTVSS (SEQ ID NO: 56); and
(f) a scFv comprising a light chain variable region that comprises a CDR1 comprising: RASQDISKYLN (SEQ ID NO: 51), a CDR2 comprising HTSRLHS (SEQ ID NO: 52); and a CDR3 comprising QQGNTLPYT (SEQ ID NO: 53) and a heavy chain variable region that comprises a CDR1 comprising: DYGVS (SEQ ID NO: 57), a CDR2 comprising VIWGSETTYYADSVKG (SEQ ID NO: 58); and a CDR3 comprising HYYYGGSYAMDY (SEQ ID NO: 59).

9. The chimeric antigen receptor of claim 8, wherein the transmembrane domain is selected from: a CD4 transmembrane domain, a CD8 transmembrane domain, or a CD28 transmembrane domain.

10. The chimeric antigen receptor of claim 8, wherein the transmembrane domain is a CD28 transmembrane domain.

11. The chimeric antigen receptor of claim 8, wherein the costimulatory domain is a CD28, 4-1BB, OX40 or a 2B4 costimulatory domain.

12. The chimeric antigen receptor of claim 8, wherein the costimulatory domain comprises the amino acid sequence of any of SEQ ID NOs:22-25 and 49.

13. The chimeric antigen receptor of claim 8, wherein the CD3ζ signaling domain comprises the amino acid sequence of SEQ ID NO:21.

14. The chimeric antigen receptor of claim 13, wherein a linker of 3 to 15 amino acids is located between the costimulatory domain and the CD3 signaling domain.

15. The chimeric antigen receptor of claim 8, wherein the spacer comprises any one of SEQ ID NOs:2-12.

16.-20. (canceled)

21. A nucleic acid molecule comprising a nucleotide sequence encoding a chimeric antigen receptor (CAR), wherein the chimeric antigen receptor comprises: an scFv targeting CD19 comprising an amino acid sequence selected from SEQ ID NO: 1 or a variant thereof having 1-5 single amino acid substitutions or the amino acid sequence of SEQ ID NO:32 or variant thereof having 1-5 single amino acid substitutions; a spacer; a transmembrane domain; a co-stimulatory domain; and a CD3ζ signaling domain.

22. The nucleic acid molecule of claim 21, encoding a polypeptide comprising a CD19 scFv comprising the amino acid sequence of SEQ ID NO:1 or a variant thereof having 1-5 single amino acid substitutions or the amino acid sequence of SEQ ID NO:32 or variant thereof having 1-5 amino acid substitutions.

23. The nucleic acid molecule of claim 21, wherein the scFv comprises the amino acid sequence of SEQ ID NO:1 or the amino acid sequence of SEQ ID NO: 32.

24. The nucleic acid molecule of claim 21, wherein the transmembrane domain is selected from: a CD4 transmembrane domain, a CD8 transmembrane domain, or a CD28 transmembrane domain.

25.-30. (canceled)

31. The nucleic acid molecule of claim 21, wherein the CAR or the polypeptide comprises the amino acid sequence of SEQ ID NO:29, 30, 31, 33, 37, 38, 39, 40, 41, 42, 43, 44, 45, 46, 47, or 48.

32.-36. (canceled)


SEQUENCE LISTING (61 sequences)

SEQ ID NO: 1 - protein, 245 aa, Artificial
  CD19 scFv VH4Vk1
DIQMTQSPSSLSASVGDRVTITCRASQDISKYLNWYQQKPGGAPKLLIYHTSRLHSGVPSRFSGSGSGTDFTFTISSLQQEDIATYYCQQGNTLPYTFGQGTKLEIKGSTSGSGKPGSGEGSTKGQVQLQESGPGLVAPSQTLSLTCTVSGVSLPDYGVSWIRQPPRKGLEWIGVIWGSETTYYNSALKSRVTISVDNSKNQFSLKLSSVTAADTAVYYCAKHYYYGGSYAMDYWGQGTLVTVSS

SEQ ID NO: 2 - protein, 10 aa, Artificial
  linker
GGGSSGGGSG

SEQ ID NO: 3 - protein, 12 aa, Artificial
  IgG4 hinge (S-->P) (S228P)
ESKYGPPCPPCP

SEQ ID NO: 4 - protein, 12 aa, Homo sapiens
ESKYGPPCPSCP

SEQ ID NO: 5 - protein, 22 aa, Artificial
  IgG4 hinge (S228P)+ linker
ESKYGPPCPPCPGGGSSGGGSG

SEQ ID NO: 6 - protein, 39 aa, Homo sapiens
IEVMYPPPYLDNEKSNGTIIHVKGKHLCPSPLFPGPSKP

SEQ ID NO: 7 - protein, 48 aa, Homo sapiens
AKPTTTPAPRPPTPAPTIASQPLSLRPEACRPAAGGAVHTRGLDFACD

SEQ ID NO: 8 - protein, 45 aa, Homo sapiens
TTTPAPRPPTPAPTIASQPLSLRPEACRPAAGGAVHTRGLDFACD

SEQ ID NO: 9 - protein, 129 aa, Artificial
  IgG4 (HL-CH3) (includes S228P in hinge)
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 229 aa, Artificial
  IgG4 (L235E, N297Q)
ESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 229 aa, Artificial
  IgG4 (S228P, L235E, N297Q)
ESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 21 aa, Homo sapiens
LCYLLDGILFIYGVILTALFL

SEQ ID NO: 14 - protein, 27 aa, Homo sapiens
FWVLVVVGGVLACYSLLVTVAFIIFWV

SEQ ID NO: 15 - protein, 28 aa, Homo sapiens
MFWVLVVVGGVLACYSLLVTVAFIIFWV

SEQ ID NO: 16 - protein, 22 aa, Homo sapiens
MALIVLGGVAGLLLFIGLGIFF

SEQ ID NO: 17 - protein, 21 aa, Homo sapiens
IYIWAPLAGTCGVLLLSLVIT

SEQ ID NO: 18 - protein, 23 aa, Homo sapiens
IYIWAPLAGTCGVLLLSLVITLY

SEQ ID NO: 19 - protein, 24 aa, Homo sapiens
IYIWAPLAGTCGVLLLSLVITLYC

SEQ ID NO: 20 - protein, 27 aa, Homo sapiens
IISFFLALTSTALLFLLFFLTLRFSVV

SEQ ID NO: 21 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 41 aa, Homo sapiens
RSKRSRLLHSDYMNMTPRRPGPTRKHYQPYAPPRDFAAYRS

SEQ ID NO: 23 - protein, 41 aa, Artificial
  CD28gg
RSKRSRGGHSDYMNMTPRRPGPTRKHYQPYAPPRDFAAYRS

SEQ ID NO: 24 - protein, 42 aa, Homo sapiens
KRGRKKLLYIFKQPFMRPVQTTQEEDGCSCRFPEEEEGGCEL

SEQ ID NO: 25 - protein, 42 aa, Homo sapiens
ALYLLRRDQRLPPDAHKPPGGGSFRTPIQEEQADAHSTLAKI

SEQ ID NO: 26 - protein, 323 aa, Homo sapiens
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 27 - protein, 24 aa, Homo sapiens
LEGGGEGRGSLLTCGDVEENPGPR

SEQ ID NO: 28 - protein, 354 aa, Homo sapiens
LVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 29 - protein, 575 aa, Artificial
  VH4Vk1-IgG4 - CD4TM-41BB with signal sequence
MLLLVTSLLLCELPHPAFLLIPDIQMTQSPSSLSASVGDRVTITCRASQDISKYLNWYQQKPGGAPKLLIYHTSRLHSGVPSRFSGSGSGTDFTFTISSLQQEDIATYYCQQGNTLPYTFGQGTKLEIKGSTSGSGKPGSGEGSTKGQVQLQESGPGLVAPSQTLSLTCTVSGVSLPDYGVSWIRQPPRKGLEWIGVIWGSETTYYNSALKSRVTISVDNSKNQFSLKLSSVTAADTAVYYCAKHYYYGGSYAMDYWGQGTLVTVSSESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 575 aa, Artificial
  4D5-IgG4 - CD4TM-41BB with signal sequence
MLLLVTSLLLCELPHPAFLLIPDIQMTQSPSSLSASVGDRVTITCRASQDISKYLNWYQQKPGKAPKLLIYHTSRLHSGVPSRFSGSRSGTDFTLTISSLQPEDFATYYCQQGNTLPYTFGQGTKVEIKGSTSGSGKPGSGEGSTKGEVQLVESGGGLVQPGGSLRLSCAASGVSLPDYGVSWVRQAPGKGLEWVAVIWGSETTYYADSVKGRFTISADTSKNTAYLQMNSLRAEDTAVYYCSRHYYYGGSYAMDYWGQGTLVTVSSESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 553 aa, Artificial
  VH4Vk1-IgG4 - CD4TM-41BB
DIQMTQSPSSLSASVGDRVTITCRASQDISKYLNWYQQKPGGAPKLLIYHTSRLHSGVPSRFSGSGSGTDFTFTISSLQQEDIATYYCQQGNTLPYTFGQGTKLEIKGSTSGSGKPGSGEGSTKGQVQLQESGPGLVAPSQTLSLTCTVSGVSLPDYGVSWIRQPPRKGLEWIGVIWGSETTYYNSALKSRVTISVDNSKNQFSLKLSSVTAADTAVYYCAKHYYYGGSYAMDYWGQGTLVTVSSESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 245 aa, Artificial
  CD19 scFv 4D5
DIQMTQSPSSLSASVGDRVTITCRASQDISKYLNWYQQKPGKAPKLLIYHTSRLHSGVPSRFSGSRSGTDFTLTISSLQPEDFATYYCQQGNTLPYTFGQGTKVEIKGSTSGSGKPGSGEGSTKGEVQLVESGGGLVQPGGSLRLSCAASGVSLPDYGVSWVRQAPGKGLEWVAVIWGSETTYYADSVKGRFTISADTSKNTAYLQMNSLRAEDTAVYYCSRHYYYGGSYAMDYWGQGTLVTVSS

SEQ ID NO: 33 - protein, 553 aa, Artificial
  4D5-IgG4 - CD4TM-41BB
DIQMTQSPSSLSASVGDRVTITCRASQDISKYLNWYQQKPGKAPKLLIYHTSRLHSGVPSRFSGSRSGTDFTLTISSLQPEDFATYYCQQGNTLPYTFGQGTKVEIKGSTSGSGKPGSGEGSTKGEVQLVESGGGLVQPGGSLRLSCAASGVSLPDYGVSWVRQAPGKGLEWVAVIWGSETTYYADSVKGRFTISADTSKNTAYLQMNSLRAEDTAVYYCSRHYYYGGSYAMDYWGQGTLVTVSSESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 4 aa, Artificial
  linker
GGGS

SEQ ID NO: 35 - protein, 5 aa, Artificial
  linker
GGGGS

SEQ ID NO: 36 - protein, 22 aa, Homo sapiens
MLLLVTSLLLCELPHPAFLLIP

SEQ ID NO: 37 - protein, 531 aa, Artificial
  VH4Vk1-IgG4 - CD4TM-41BB
DIQMTQSPSSLSASVGDRVTITCRASQDISKYLNWYQQKPGGAPKLLIYHTSRLHSGVPSRFSGSGSGTDFTFTISSLQQEDIATYYCQQGNTLPYTFGQGTKLEIKGSTSGSGKPGSGEGSTKGQVQLQESGPGLVAPSQTLSLTCTVSGVSLPDYGVSWIRQPPRKGLEWIGVIWGSETTYYNSALKSRVTISVDNSKNQFSLKLSSVTAADTAVYYCAKHYYYGGSYAMDYWGQGTLVTVSSGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 553 aa, Artificial
  VH4Vk1-IgG4 - CD4TM-41BB with signal sequence
MLLLVTSLLLCELPHPAFLLIPDIQMTQSPSSLSASVGDRVTITCRASQDISKYLNWYQQKPGGAPKLLIYHTSRLHSGVPSRFSGSGSGTDFTFTISSLQQEDIATYYCQQGNTLPYTFGQGTKLEIKGSTSGSGKPGSGEGSTKGQVQLQESGPGLVAPSQTLSLTCTVSGVSLPDYGVSWIRQPPRKGLEWIGVIWGSETTYYNSALKSRVTISVDNSKNQFSLKLSSVTAADTAVYYCAKHYYYGGSYAMDYWGQGTLVTVSSGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 531 aa, Artificial
  4D5-IgG4 - CD4TM-41BB
DIQMTQSPSSLSASVGDRVTITCRASQDISKYLNWYQQKPGKAPKLLIYHTSRLHSGVPSRFSGSRSGTDFTLTISSLQPEDFATYYCQQGNTLPYTFGQGTKVEIKGSTSGSGKPGSGEGSTKGEVQLVESGGGLVQPGGSLRLSCAASGVSLPDYGVSWVRQAPGKGLEWVAVIWGSETTYYADSVKGRFTISADTSKNTAYLQMNSLRAEDTAVYYCSRHYYYGGSYAMDYWGQGTLVTVSSGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 553 aa, Artificial
  4D5-IgG4 - CD4TM-41BB with signal sequence
MLLLVTSLLLCELPHPAFLLIPDIQMTQSPSSLSASVGDRVTITCRASQDISKYLNWYQQKPGKAPKLLIYHTSRLHSGVPSRFSGSRSGTDFTLTISSLQPEDFATYYCQQGNTLPYTFGQGTKVEIKGSTSGSGKPGSGEGSTKGEVQLVESGGGLVQPGGSLRLSCAASGVSLPDYGVSWVRQAPGKGLEWVAVIWGSETTYYADSVKGRFTISADTSKNTAYLQMNSLRAEDTAVYYCSRHYYYGGSYAMDYWGQGTLVTVSSGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 552 aa, Artificial
  VH4Vk1-IgG4 - CD8TM-41BB
DIQMTQSPSSLSASVGDRVTITCRASQDISKYLNWYQQKPGGAPKLLIYHTSRLHSGVPSRFSGSGSGTDFTFTISSLQQEDIATYYCQQGNTLPYTFGQGTKLEIKGSTSGSGKPGSGEGSTKGQVQLQESGPGLVAPSQTLSLTCTVSGVSLPDYGVSWIRQPPRKGLEWIGVIWGSETTYYNSALKSRVTISVDNSKNQFSLKLSSVTAADTAVYYCAKHYYYGGSYAMDYWGQGTLVTVSSESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 574 aa, Artificial
  VH4Vk1-IgG4 - CD8TM-41BB with signal sequence
MLLLVTSLLLCELPHPAFLLIPDIQMTQSPSSLSASVGDRVTITCRASQDISKYLNWYQQKPGGAPKLLIYHTSRLHSGVPSRFSGSGSGTDFTFTISSLQQEDIATYYCQQGNTLPYTFGQGTKLEIKGSTSGSGKPGSGEGSTKGQVQLQESGPGLVAPSQTLSLTCTVSGVSLPDYGVSWIRQPPRKGLEWIGVIWGSETTYYNSALKSRVTISVDNSKNQFSLKLSSVTAADTAVYYCAKHYYYGGSYAMDYWGQGTLVTVSSESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 552 aa, Artificial
  4D5-IgG4 - CD8TM-41BB
DIQMTQSPSSLSASVGDRVTITCRASQDISKYLNWYQQKPGKAPKLLIYHTSRLHSGVPSRFSGSRSGTDFTLTISSLQPEDFATYYCQQGNTLPYTFGQGTKVEIKGSTSGSGKPGSGEGSTKGEVQLVESGGGLVQPGGSLRLSCAASGVSLPDYGVSWVRQAPGKGLEWVAVIWGSETTYYADSVKGRFTISADTSKNTAYLQMNSLRAEDTAVYYCSRHYYYGGSYAMDYWGQGTLVTVSSESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 574 aa, Artificial
  4D5-IgG4 -CD8TM-41BB with signal sequence
MLLLVTSLLLCELPHPAFLLIPDIQMTQSPSSLSASVGDRVTITCRASQDISKYLNWYQQKPGKAPKLLIYHTSRLHSGVPSRFSGSRSGTDFTLTISSLQPEDFATYYCQQGNTLPYTFGQGTKVEIKGSTSGSGKPGSGEGSTKGEVQLVESGGGLVQPGGSLRLSCAASGVSLPDYGVSWVRQAPGKGLEWVAVIWGSETTYYADSVKGRFTISADTSKNTAYLQMNSLRAEDTAVYYCSRHYYYGGSYAMDYWGQGTLVTVSSESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 530 aa, Artificial
  VH4Vk1-IgG4 -CD8TM-41BB
DIQMTQSPSSLSASVGDRVTITCRASQDISKYLNWYQQKPGGAPKLLIYHTSRLHSGVPSRFSGSGSGTDFTFTISSLQQEDIATYYCQQGNTLPYTFGQGTKLEIKGSTSGSGKPGSGEGSTKGQVQLQESGPGLVAPSQTLSLTCTVSGVSLPDYGVSWIRQPPRKGLEWIGVIWGSETTYYNSALKSRVTISVDNSKNQFSLKLSSVTAADTAVYYCAKHYYYGGSYAMDYWGQGTLVTVSSGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 552 aa, Artificial
  VH4Vk1-IgG4 - CD8TM-41BB
MLLLVTSLLLCELPHPAFLLIPDIQMTQSPSSLSASVGDRVTITCRASQDISKYLNWYQQKPGGAPKLLIYHTSRLHSGVPSRFSGSGSGTDFTFTISSLQQEDIATYYCQQGNTLPYTFGQGTKLEIKGSTSGSGKPGSGEGSTKGQVQLQESGPGLVAPSQTLSLTCTVSGVSLPDYGVSWIRQPPRKGLEWIGVIWGSETTYYNSALKSRVTISVDNSKNQFSLKLSSVTAADTAVYYCAKHYYYGGSYAMDYWGQGTLVTVSSGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 530 aa, Artificial
  4D5-IgG4 - CD8TM-41BB
DIQMTQSPSSLSASVGDRVTITCRASQDISKYLNWYQQKPGKAPKLLIYHTSRLHSGVPSRFSGSRSGTDFTLTISSLQPEDFATYYCQQGNTLPYTFGQGTKVEIKGSTSGSGKPGSGEGSTKGEVQLVESGGGLVQPGGSLRLSCAASGVSLPDYGVSWVRQAPGKGLEWVAVIWGSETTYYADSVKGRFTISADTSKNTAYLQMNSLRAEDTAVYYCSRHYYYGGSYAMDYWGQGTLVTVSSGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 552 aa, Artificial
  4D5-IgG4 -CD8TM-41BB with signal sequence
MLLLVTSLLLCELPHPAFLLIPDIQMTQSPSSLSASVGDRVTITCRASQDISKYLNWYQQKPGKAPKLLIYHTSRLHSGVPSRFSGSRSGTDFTLTISSLQPEDFATYYCQQGNTLPYTFGQGTKVEIKGSTSGSGKPGSGEGSTKGEVQLVESGGGLVQPGGSLRLSCAASGVSLPDYGVSWVRQAPGKGLEWVAVIWGSETTYYADSVKGRFTISADTSKNTAYLQMNSLRAEDTAVYYCSRHYYYGGSYAMDYWGQGTLVTVSSGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 120 aa, Homo sapiens
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 50 - protein, 110 aa, Artificial
  CD19 scFv light chain variable region
DIQMTQSPSSLSASVGDRVTITCRASQDISKYLNWYQQKPGGAPKLLIYHTSRLHSGVPSRFSGSGSGTDFTFTISSLQQEDIATYYCQQGNTLPYTFGQGTKLEIKGST

SEQ ID NO: 51 - protein, 11 aa, Artificial
  CD19 scFv CDR1
RASQDISKYLN

SEQ ID NO: 52 - protein, 7 aa, Artificial
  CD19 scFv CDR2
HTSRLHS

SEQ ID NO: 53 - protein, 9 aa, Artificial
  CD19 scFv CDR3
QQGNTLPYT

SEQ ID NO: 54 - protein, 120 aa, Artificial
  CD19 scFv heavy chain variable region
QVQLQESGPGLVAPSQTLSLTCTVSGVSLPDYGVSWIRQPPRKGLEWIGVIWGSETTYYNSALKSRVTISVDNSKNQFSLKLSSVTAADTAVYYCAKHYYYGGSYAMDYWGQGTLVTVSS

SEQ ID NO: 55 - protein, 5 aa, Artificial
  CD19 scFv CDR1
DYGVS

SEQ ID NO: 56 - protein, 16 aa, Artificial
  CD19 scFv CDR2
VIWGSETTYYNSALKS

SEQ ID NO: 57 - protein, 12 aa, Artificial
  CD19 scFv CDR3
HYYYGGSYAMDY

SEQ ID NO: 58 - protein, 110 aa, Artificial
  CD19 scFv light chain variable region
DIQMTQSPSSLSASVGDRVTITCRASQDISKYLNWYQQKPGKAPKLLIYHTSRLHSGVPSRFSGSRSGTDFTLTISSLQPEDFATYYCQQGNTLPYTFGQGTKVEIKGST

SEQ ID NO: 59 - protein, 120 aa, Artificial
  CD19 scFv heavy chain variable region
EVQLVESGGGLVQPGGSLRLSCAASGVSLPDYGVSWVRQAPGKGLEWVAVIWGSETTYYADSVKGRFTISADTSKNTAYLQMNSLRAEDTAVYYCSRHYYYGGSYAMDYWGQGTLVTVSS

SEQ ID NO: 60 - protein, 16 aa, Artificial
  CD19 scFv CDR2
VIWGSETTYYADSVKG

SEQ ID NO: 61 - protein, 21 aa, Homo sapiens
PFFFCCFIAVAMGIRFIIMVA